SEQUENCE LISTING (65 sequences)

SEQ ID NO: 1 - DNA, 34 bp, Artificial Sequence
  A synthetic primer
cgaggatccgccatggagcttctatcgccgccac

SEQ ID NO: 2 - DNA, 33 bp, Artificial Sequence
  A synthetic primer
cttttttctccccatgtgctcctccggtttcag

SEQ ID NO: 3 - DNA, 33 bp, Artificial Sequence
  A synthetic primer
ctgaaaccggaggagcacatggggagaaaaaag

SEQ ID NO: 4 - DNA, 30 bp, Artificial Sequence
  A synthetic primer
cgctcgagtctcatgttgcccatccttcag

SEQ ID NO: 5 - DNA, 28 bp, Artificial Sequence
  A synthetic primer
cgaggatccatggagctactgtcgccac

SEQ ID NO: 6 - DNA, 37 bp, Artificial Sequence
  A synthetic primer
gaatcttttttctccccatgtgctcttcgggtttcag

SEQ ID NO: 7 - DNA, 37 bp, Artificial Sequence
  A synthetic primer
ctgaaacccgaagagcacatggggagaaaaaagattc

SEQ ID NO: 8 - DNA, 27 bp, Artificial Sequence
  A synthetic primer
cgactcgagttatgttgcccatccttc

SEQ ID NO: 9 - DNA, 35 bp, Artificial Sequence
  A synthetic primer
ctgaaggatgggcaacaatggagcttctatcgccg

SEQ ID NO: 10 - DNA, 36 bp, Artificial Sequence
  A synthetic primer
cgactcgaggaattctcagtgctcctccggtttcag

SEQ ID NO: 11 - DNA, 26 bp, Artificial Sequence
  A synthetic primer
gaggatccgccatggggagaaaaaag

SEQ ID NO: 12 - DNA, 35 bp, Artificial Sequence
  A synthetic primer
cggcgatagaagctccattgttgcccatccttcag

SEQ ID NO: 13 - DNA, 34 bp, Artificial Sequence
  A synthetic primer
ctgaaggatgggcaacaatggagctactgtcgcc

SEQ ID NO: 14 - DNA, 36 bp, Artificial Sequence
  A synthetic primer
cgactcgaggaattctcagtgctcttcgggtttcag

SEQ ID NO: 15 - DNA, 35 bp, Artificial Sequence
  A synthetic primer
tggcgacagtagctccattgttgcccatccttcag

SEQ ID NO: 16 - DNA, 41 bp, Artificial Sequence
  A synthetic primer
cgaggatccgaattcctcgagatgttgggggacggggattc

SEQ ID NO: 17 - DNA, 34 bp, Artificial Sequence
  A synthetic primer
cgagcggccgctcacccaccgtactcgtcaattc

SEQ ID NO: 18 - DNA, 26 bp, Artificial Sequence
  A synthetic primer
cgactcgagtgttgcccatccttcag

SEQ ID NO: 19 - DNA, 33 bp, Artificial Sequence
  A synthetic primer
gagaattcgccatggagcttctatcgccgccac

SEQ ID NO: 20 - DNA, 36 bp, Artificial Sequence
  A synthetic primer
ggccaggctttggtacatgtgctcctccggtttcag

SEQ ID NO: 21 - DNA, 36 bp, Artificial Sequence
  A synthetic primer
ctgaaaccggaggagcacatgtaccaaagcctggcc

SEQ ID NO: 22 - DNA, 28 bp, Artificial Sequence
  A synthetic primer
cggaattctcttacgcggtgattatgtc

SEQ ID NO: 23 - DNA, 43 bp, Artificial Sequence
  A synthetic primer
cgaggatccgaattcctcgagatggagcttctatcgccgccac

SEQ ID NO: 24 - DNA, 32 bp, Artificial Sequence
  A synthetic primer
cgagcggccgctcagtgctcctccggtttcag

SEQ ID NO: 25 - DNA, 30 bp, Artificial Sequence
  A synthetic primer
cgaggatccgccatgtaccaaagcctggcc

SEQ ID NO: 26 - DNA, 24 bp, Artificial Sequence
  A synthetic primer
cgactcgagcgcggtgattatgtc

SEQ ID NO: 27 - DNA, 34 bp, Artificial Sequence; identical to SEQ ID NO: 1
  A synthetic primer

SEQ ID NO: 28 - DNA, 33 bp, Artificial Sequence
  A synthetic primer
cgactcgaggaattcgtgctcctccggtttcag

SEQ ID NO: 29 - DNA, 31 bp, Artificial Sequence
  A synthetic primer
cgagaattcatgagtctggtggggggctttc

SEQ ID NO: 30 - DNA, 30 bp, Artificial Sequence
  A synthetic primer
gcgctcgagctactgcttgagctccagggc

SEQ ID NO: 31 - DNA, 34 bp, Artificial Sequence
  A synthetic primer
cgaggatccgccatgagtctggtggggggctttc

SEQ ID NO: 32 - DNA, 27 bp, Artificial Sequence
  A synthetic primer
cgactcgagctgcttgagctccagggc

SEQ ID NO: 33 - DNA, 36 bp, Artificial Sequence
  A synthetic primer
ctcatctgtatcggccatgtgctcctccggtttcag

SEQ ID NO: 34 - DNA, 36 bp, Artificial Sequence
  A synthetic primer
ctgaaaccggaggagcacatggccgatacagatgag

SEQ ID NO: 35 - DNA, 29 bp, Artificial Sequence
  A synthetic primer
cggaattctcttagctattctcactccac

SEQ ID NO: 36 - DNA, 27 bp, Artificial Sequence
  A synthetic primer
cgaggatccgccatggccgatacagag

SEQ ID NO: 37 - DNA, 25 bp, Artificial Sequence
  A synthetic primer
cgactcgaggctattctcactccac

SEQ ID NO: 38 - DNA, 28 bp, Artificial Sequence
  A synthetic primer
cgaggatccatggagctactgtcgccac

SEQ ID NO: 39 - DNA, 33 bp, Artificial Sequence
  A synthetic primer
cgactcgaggaattcgtgctcttcgggtttcag

SEQ ID NO: 40 - DNA, 30 bp, Artificial Sequence
  A synthetic primer
cgagaattcatgaatgatttcggaatcaag

SEQ ID NO: 41 - DNA, 28 bp, Artificial Sequence
  A synthetic primer
cgactcgagtcagtcctccgtgtcgggc

SEQ ID NO: 42 - DNA, 30 bp, Artificial Sequence
  A synthetic primer
cgagaattcatggcccagcccctgtgcccg

SEQ ID NO: 43 - DNA, 28 bp, Artificial Sequence
  A synthetic primer
cgactcgagtcacttgggctcctcaggc

SEQ ID NO: 44 - DNA, 29 bp, Artificial Sequence
  A synthetic primer
cgaggatccatgttgggggacggggattc

SEQ ID NO: 45 - DNA, 30 bp, Artificial Sequence
  A synthetic primer
cttttttctccccatcccaccgtactcgtc

SEQ ID NO: 46 - DNA, 30 bp, Artificial Sequence
  A synthetic primer
gacgagtacggtgggatggggagaaaaaag

SEQ ID NO: 47 - DNA, 1491 bp, Mus musculus
atggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcacatggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacatga

SEQ ID NO: 48 - DNA, 1608 bp, Homo sapiens
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcacatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacataa

SEQ ID NO: 49 - DNA, 1491 bp, Mus musculus
atggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacaatggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcactga

SEQ ID NO: 50 - DNA, 1608 bp, Homo sapiens
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacaatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactga

SEQ ID NO: 51 - DNA, 1446 bp, Mus musculus
atggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacactcgagatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 52 - DNA, 1563 bp, Homo sapiens
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacactcgagatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 53 - DNA, 1512 bp, Mus musculus
atggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcacatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaa

SEQ ID NO: 54 - DNA, 1518 bp, Mus musculus
atgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgctcgagatggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcactga

SEQ ID NO: 55 - DNA, 1467 bp, Mus musculus
atgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgctcgagatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 56 - DNA, 846 bp, Mus musculus
atggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcacgaattcatgagtctggtggggggctttccccaccaccccgtggtgcaccatgagggctacccattcgccgcagccgccgccgccgctgccgccgccgccgccagccgctgcagccacgaggagaacccctacttccacggctggcttattggccacccggagatgtcgccccccgactacagtatggccctgtcctacagccccgagtacgccagcggtgccgcgggcctggaccactcccattatgggggagtgccgcccggcgccgggcctcccggcctgggggggccgcgcccggtgaagcgccggggcaccgccaaccgcaaggagcggcgcaggactcagagcatcaacagcgccttcgccgagctgcgcgagtgcatccccaacgtgcccgccgacaccaaactctccaagatcaagacactgcgcctggccaccagctacatcgcctacctcatggatctgctggccaaggacgaccagaacggagaggcggaggccttcaaggcggagatcaagaagaccgacgtgaaagaggagaagaggaagaaagagctgaatgagatcttgaaaagcacagtgagcagcaacgacaagaaaaccaaaggccggacaggctggccacagcacgtctgggccctggagctcaagcagtga

SEQ ID NO: 57 - DNA, 846 bp, Mus musculus
atgagtctggtggggggctttccccaccaccccgtggtgcaccatgagggctacccattcgccgcagccgccgccgccgctgccgccgccgccgccagccgctgcagccacgaggagaacccctacttccacggctggcttattggccacccggagatgtcgccccccgactacagtatggccctgtcctacagccccgagtacgccagcggtgccgcgggcctggaccactcccattatgggggagtgccgcccggcgccgggcctcccggcctgggggggccgcgcccggtgaagcgccggggcaccgccaaccgcaaggagcggcgcaggactcagagcatcaacagcgccttcgccgagctgcgcgagtgcatccccaacgtgcccgccgacaccaaactctccaagatcaagacactgcgcctggccaccagctacatcgcctacctcatggatctgctggccaaggacgaccagaacggagaggcggaggccttcaaggcggagatcaagaagaccgacgtgaaagaggagaagaggaagaaagagctgaatgagatcttgaaaagcacagtgagcagcaacgacaagaaaaccaaaggccggacaggctggccacagcacgtctgggccctggagctcaagcagctcgagatggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcactga

SEQ ID NO: 58 - DNA, 795 bp, Mus musculus
atgagtctggtggggggctttccccaccaccccgtggtgcaccatgagggctacccattcgccgcagccgccgccgccgctgccgccgccgccgccagccgctgcagccacgaggagaacccctacttccacggctggcttattggccacccggagatgtcgccccccgactacagtatggccctgtcctacagccccgagtacgccagcggtgccgcgggcctggaccactcccattatgggggagtgccgcccggcgccgggcctcccggcctgggggggccgcgcccggtgaagcgccggggcaccgccaaccgcaaggagcggcgcaggactcagagcatcaacagcgccttcgccgagctgcgcgagtgcatccccaacgtgcccgccgacaccaaactctccaagatcaagacactgcgcctggccaccagctacatcgcctacctcatggatctgctggccaaggacgaccagaacggagaggcggaggccttcaaggcggagatcaagaagaccgacgtgaaagaggagaagaggaagaaagagctgaatgagatcttgaaaagcacagtgagcagcaacgacaagaaaaccaaaggccggacaggctggccacagcacgtctgggccctggagctcaagcagctcgagatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 59 - DNA, 1743 bp, Mus musculus
atggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcacatggccgatacagatgagggctttggcctggcgcgcacgcctctggagcctgattccaaagacaggtcttgcgattcgaaacctgagagtgctctgggggctcccagcaagtctccatcatccccgcaggctgccttcacccagcagggcatggaaggaatcaaggtgtttcttcatgaacgtgaactgtggctgaagttccacgaagtgggcacagagatgatcatcaccaaggcagggaggagaatgtttcctagttacaaagtgaaggtgactggccttaatcccaaaacgaagtatattcttctcatggatattgttcccgcagacgaccacagatataaatttgctgataacaaatggtccgtaactggcaaagcagagcctgccatgccggggcgcctttacgtgcacccggactccccagcaaccggagcccactggatgcgacaacttgtctccttccagaagctcaaactcaccaacaaccacctggacccgtttggacacattatcctgaactccatgcacaaataccagccccgattacacatcgtgaaagcagacgaaaataatgggttcggttcaaagaacactgcgttttgcacccacgtcttcccggagacagcttttatcgctgtgacttcgtaccagaatcacaagatcacacagctgaaaattgagaacaaccccttcgccaaaggctttcggggcagtgatgacctggagttacacaggatgtctcggatgcaaagtaaagagtatcctgtggttcccaggagcacagtgaggcacaaagtcacctccaaccacagccccttcagcagcgagacccgagctctctccacctcatccaatttagggtcccagtaccagtgtgagaatggtgtctctggcccctcccaggaccttctgcccccacctaacccatacccactggcccaggagcacagccaaatttaccactgtaccaagaggaaagatgaggaatgttccagcacggagcacccctataagaagccgtacatggagacatcccccagcgaggaagacaccttctatcgctcgggctacccccagcagcagggcctgagtacctcttacaggacagagtcggcccagcggcaggcctgcatgtatgccagctccgctccccccagcgagcccgtgcctagcctggaggacatcagctgtaacacatggcccagcatgccctcctatagcagctgtaccgtcaccaccgtgcagcccatggaccgtcttccctaccagcacttctccgctcatttcacctcggggcccctggtccctcggttggctggcatggccaaccatggttctccccagctcggcgaagggatgtttcagcaccagacctcagtggcccatcagcctgtggtcaggcagtgcgggcctcagactggccttcagtctccgggcggcctccagcccccagagtttctctacactcacggcgtgcccaggaccctgtccccccatcagtatcactcggtacacggcgtcggcatggtgccagagtggagtgagaatagctaa

SEQ ID NO: 60 - DNA, 1749 bp, Mus musculus
atggccgatacagatgagggctttggcctggcgcgcacgcctctggagcctgattccaaagacaggtcttgcgattcgaaacctgagagtgctctgggggctcccagcaagtctccatcatccccgcaggctgccttcacccagcagggcatggaaggaatcaaggtgtttcttcatgaacgtgaactgtggctgaagttccacgaagtgggcacagagatgatcatcaccaaggcagggaggagaatgtttcctagttacaaagtgaaggtgactggccttaatcccaaaacgaagtatattcttctcatggatattgttcccgcagacgaccacagatataaatttgctgataacaaatggtccgtaactggcaaagcagagcctgccatgccggggcgcctttacgtgcacccggactccccagcaaccggagcccactggatgcgacaacttgtctccttccagaagctcaaactcaccaacaaccacctggacccgtttggacacattatcctgaactccatgcacaaataccagccccgattacacatcgtgaaagcagacgaaaataatgggttcggttcaaagaacactgcgttttgcacccacgtcttcccggagacagcttttatcgctgtgacttcgtaccagaatcacaagatcacacagctgaaaattgagaacaaccccttcgccaaaggctttcggggcagtgatgacctggagttacacaggatgtctcggatgcaaagtaaagagtatcctgtggttcccaggagcacagtgaggcacaaagtcacctccaaccacagccccttcagcagcgagacccgagctctctccacctcatccaatttagggtcccagtaccagtgtgagaatggtgtctctggcccctcccaggaccttctgcccccacctaacccatacccactggcccaggagcacagccaaatttaccactgtaccaagaggaaagatgaggaatgttccagcacggagcacccctataagaagccgtacatggagacatcccccagcgaggaagacaccttctatcgctcgggctacccccagcagcagggcctgagtacctcttacaggacagagtcggcccagcggcaggcctgcatgtatgccagctccgctccccccagcgagcccgtgcctagcctggaggacatcagctgtaacacatggcccagcatgccctcctatagcagctgtaccgtcaccaccgtgcagcccatggaccgtcttccctaccagcacttctccgctcatttcacctcggggcccctggtccctcggttggctggcatggccaaccatggttctccccagctcggcgaagggatgtttcagcaccagacctcagtggcccatcagcctgtggtcaggcagtgcgggcctcagactggccttcagtctccgggcggcctccagcccccagagtttctctacactcacggcgtgcccaggaccctgtccccccatcagtatcactcggtacacggcgtcggcatggtgccagagtggagtgagaatagcctcgagatggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcactga

SEQ ID NO: 61 - DNA, 1698 bp, Mus musculus
atggccgatacagatgagggctttggcctggcgcgcacgcctctggagcctgattccaaagacaggtcttgcgattcgaaacctgagagtgctctgggggctcccagcaagtctccatcatccccgcaggctgccttcacccagcagggcatggaaggaatcaaggtgtttcttcatgaacgtgaactgtggctgaagttccacgaagtgggcacagagatgatcatcaccaaggcagggaggagaatgtttcctagttacaaagtgaaggtgactggccttaatcccaaaacgaagtatattcttctcatggatattgttcccgcagacgaccacagatataaatttgctgataacaaatggtccgtaactggcaaagcagagcctgccatgccggggcgcctttacgtgcacccggactccccagcaaccggagcccactggatgcgacaacttgtctccttccagaagctcaaactcaccaacaaccacctggacccgtttggacacattatcctgaactccatgcacaaataccagccccgattacacatcgtgaaagcagacgaaaataatgggttcggttcaaagaacactgcgttttgcacccacgtcttcccggagacagcttttatcgctgtgacttcgtaccagaatcacaagatcacacagctgaaaattgagaacaaccccttcgccaaaggctttcggggcagtgatgacctggagttacacaggatgtctcggatgcaaagtaaagagtatcctgtggttcccaggagcacagtgaggcacaaagtcacctccaaccacagccccttcagcagcgagacccgagctctctccacctcatccaatttagggtcccagtaccagtgtgagaatggtgtctctggcccctcccaggaccttctgcccccacctaacccatacccactggcccaggagcacagccaaatttaccactgtaccaagaggaaagatgaggaatgttccagcacggagcacccctataagaagccgtacatggagacatcccccagcgaggaagacaccttctatcgctcgggctacccccagcagcagggcctgagtacctcttacaggacagagtcggcccagcggcaggcctgcatgtatgccagctccgctccccccagcgagcccgtgcctagcctggaggacatcagctgtaacacatggcccagcatgccctcctatagcagctgtaccgtcaccaccgtgcagcccatggaccgtcttccctaccagcacttctccgctcatttcacctcggggcccctggtccctcggttggctggcatggccaaccatggttctccccagctcggcgaagggatgtttcagcaccagacctcagtggcccatcagcctgtggtcaggcagtgcgggcctcagactggccttcagtctccgggcggcctccagcccccagagtttctctacactcacggcgtgcccaggaccctgtccccccatcagtatcactcggtacacggcgtcggcatggtgccagagtggagtgagaatagcctcgagatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 62 - DNA, 1602 bp, Homo sapiens
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcacgaattcatgaatgatttcggaatcaagaatatggaccaggtagcccctgtggctaacagttacagagggacactcaagcgccagccagcctttgacacctttgatgggtccctgtttgctgtttttccttctctaaatgaagagcaaacactgcaagaagtgccaacaggcttggattccatttctcatgactccgccaactgtgaattgcctttgttaaccccgtgcagcaaggctgtgatgagtcaagccttaaaagctaccttcagtggcttcaaaaaggaacagcggcgcctgggcattccaaagaacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaagaaaaccaagaaaagacagaagatcaatatgaagaaaattcacacctcacctccgttcctcattggattaacagcaatacattaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctgggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggaagtggacctattcagctgtggcagtttctcctggagctgctatcagacaaatcctgccagtcattcatcagctggactggagacggatgggagtttaagctcgccgaccccgatgaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactga

SEQ ID NO: 63 - DNA, 999 bp, Homo sapiens
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcacgaattcatggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgccctgaagggcaggcgatggagccaagcccaccgtccccgctccttccgggcgacgtgctggctctgttggagacctggatgcccctctcgcctctggagtggctgcctgaggagcccaagtga

SEQ ID NO: 64 - DNA, 1536 bp, Mus musculus
atgttgggggacggggattccccgggtccgggatttaccccccacgactccgccccctacggcgctctggatatggccgacttcgagtttgagcagatgtttaccgatgcccttggaattgacgagtacggtgggatggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagcttgcactagcactcatttatctcagagttcaaatctctccctgccttctactcaaagcctcagcatcaagtcagaacctgtttctcctcctagagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacatga

SEQ ID NO: 65 - DNA, 1557 bp, Homo sapiens
atgttgggggacggggattccccgggtccgggatttaccccccacgactccgccccctacggcgctctggatatggccgacttcgagtttgagcagatgtttaccgatgcccttggaattgacgagtacggtgggatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacataa

(19) United States
(12) Patent Application Publication
(10) Pub. No.: US 2014/0377236 A1
(43) Pub. Date: Dec. 25, 2014

(54) METHOD OF EFFICIENTLY CONVERTING NON-CARDIAC CELLS INTO CARDIOVASCULAR CELLS

(71) Applicants: Hiroyuki Hirai, Minneapolis, MN (US); Nobuaki Kikyo, Edina, MN (US)

(72) Inventors: Hiroyuki Hirai, Minneapolis, MN (US); Nobuaki Kikyo, Edina, MN (US)

(21) Appl. No.: 14/310,730

(22) Filed: Jun. 20, 2014

Related U.S. Application Data
(60) Provisional application No. 61/837,617, filed on Jun. 20, 2013.

(51) Int. Cl. C12N 15/85

(52) U.S. Cl. CPC C12N 15/85
USPC 424/93.21; 435/455; 514/44 R

(57) ABSTRACT

Described herein is a method for generating cardiomyocytes (CMs) from non-cardiac cells.

### CROSS-REFERENCE TO RELATED APPLICATIONS

The present application claims the priority benefits of U.S. Provisional Application No. 61/837,617, filed Jun. 20, 2013, which is expressly incorporated fully herein by reference.

### STATEMENT OF GOVERNMENT RIGHTS

This invention was made with government support under United States Grants No. R01 DK082430 and No. R01 GM098294 from the National Institutes of Health. The government has certain rights in the invention.

### BACKGROUND OF THE INVENTION

Ischemic heart disease is a leading cause of death in adults in developed countries. This is primarily because cardiac tissues cannot regenerate and replace damaged tissues. Most cardiomyocytes (CMs) are terminally differentiated and stop dividing shortly after birth. A promising strategy for repairing damaged cardiac tissues is transplantation of healthy CMs. Ieda et al. reported in 2010 a new strategy to convert fibroblasts into CMs by introducing three CM-specific transcription factor genes—Gata4, Mef2c and Tbx5. This strategy can convert heart-derived fibroblasts, but not skin fibroblasts, more useful cells for medical applications. To overcome this limitation, Song et al. added another factor Hand2 and succeeded to convert fibroblasts isolated from mouse tails to CMs. This conversion required the conditioned medium prepared from CMs. In addition, the efficiency of making beating CMs was extremely low (0.0036%).

### SUMMARY OF THE INVENTION

The present invention provides a method for improving the efficiency of generating cardiomyocytes (CMs) directly from non-cardiac cells (improved direct reprogramming of cells (e.g., fibroblast cells) to cardiomyocytes (e.g., beating cardiomyocytes) with a transactivation domain (e.g., obtained from MyoD) fused to a transcription factor (e.g., a cardiac transcription factor). Although direct reprogramming of non-cardiac cells into iCMs provides a novel strategy to prepare CMs for transplantation, previously reported protocols are highly inefficient. This is a major problem for clinical application of the strategy because a large number of CMs, which do not proliferate, cannot be prepared with this approach. Fusion of the powerful transcription activation domain of the MyoD and VP16 proteins to the Mef2c protein or other cardiac proteins, including Gata4, Tbx5, Hand2, Ets2, and Mesp1, converts fibroblasts to iCMs faster and more efficiently than previously reported methods using corresponding wild-type proteins. In principle, a transcription activation domain of any transcription factors can be combined with any heterologous transcription factors, including ubiquitously expressed factors such as c-Myc. This improvement will substantially facilitate realization of this strategy. In addition the compositions described herein, e.g., Mef2cM₃-GHT, can be directly injected around the infarct area to induce regeneration of iCMs from non-cardiac cells in situ

One embodiment provides a method for preparing a cardiomyocyte (CM; cells that make up cardiac muscle) comprising introducing a nucleic acid sequence which is a fusion (created through the joining of two or more genes of fragments thereof) of a heterologous transactivation domain and a transcription factor into a somatic cell (for example, a non-germ line cell or a cell which is not a stem cell). In one embodiment, the fusion is a DNA, RNA or protein. In one embodiment, the fusion is introduced to the cell in vivo or in vitro. In another embodiment, the transcription factor is Gata4, Mef2c, Tbx5, Hand2, Ets2, or Mesp1. In one embodiment, the transactivation domain is obtained from MyoD or VP16, for example, the transactivation domain can be obtained from MyoD (e.g., the N-terminus region of MyoD, including the region coding for or comprising amino acids 1-62 of MyoD or a region/nucleic acid sequence that is at least about 80% identical thereto.

In one embodiment, an additional further factor is introduced into the cell, wherein the additional factor comprises Mef2cM₃, Mef2cVP16, M₃Gata4, M₃Hand2, M₃Tbx5, M₃Ets2, M₃Mesp1, Mef2c, Gata4, Hand2, Tbx5, Ets2, and/or Mesp1.

In one embodiment, Mef2cM₃, Gata4, Hand2, and Tbx5; Mef2c, M₃Gata4, Hand2, and Tbx5; Mef2c, Gata4, M₃Hand2, and Tbx5; Mef2c, Gata4, Hand2, and M₃Tbx5; Mef2cM₃, M₃Gata4, Hand2, and Tbx5; Mef2cM₃, Gata4, M₃Hand2, and Tbx5; Mef2cM₃, Gata4, Hand2, and M₃Tbx5; Mef2c, M₃Gata4, M₃Hand2, and Tbx5; Mef2c, M₃Gata4, Hand2, and M₃Tbx5; Mef2c, Gata4, M₃Hand2, and M₃Tbx5; Mef2cM₃, M₃Gata4, M₃Hand2, and Tbx5; Mef2cM₃, M₃Gata4, Hand2, and M₃Tbx5; Mef2cM₃, Gata4, M₃Hand2, and M₃Tbx5; Mef2c, M₃Gata4, M₃Hand2, and M₃Tbx5; Mef2cM₃, M₃Gata4, M₃Hand2, and M₃Tbx5; Mef2cM₃, M₃Gata4, M₃Hand2, M₃Tbx5, Mef2c, Gata4, Hand2, and Tbx5; Mef2cM₃, M₃Gata4, M₃Hand2, M₃Tbx5, M₃Ets2, and M₃Mesp1; Mef2cM₃, M₃Gata4, M₃Hand2, M₃Tbx5, Mef2c, Gata4, Hand2, Tbx5, Ets2, and Mesp1; Mef2cM₃, M₃Gata4, M₃Hand2, M₃Tbx5, M₃Ets2, M₃Mesp1, Mef2c, Gata4, Hand2, Tbx5, Ets2, and Mesp1; Mef2cVP16, Gata4, Hand2, and Tbx5; Mef2c, M₃Gata4, Hand2, and Tbx5; Mef2c, Gata4, M₃Hand2, and Tbx5; Mef2c, Gata4, Hand2, and M₃Tbx5; Mef2cVP16, M₃Gata4, Hand2, and Tbx5; Mef2cVP16, Gata4, M₃Hand2, and Tbx5; Mef2cVP16, Gata4, Hand2, and M₃Tbx5; Mef2c, M₃Gata4, M₃Hand2, and Tbx5; Mef2c, M₃Gata4, Hand2, and M₃Tbx5; Mef2c, Gata4, M₃Hand2, and M₃Tbx5; Mef2cVP16, M₃Gata4, M₃Hand2, and Tbx5; Mef2cVP16, M₃Gata4, Hand2, and M₃Tbx5; Mef2cVP16, Gata4, M₃Hand2, and M₃Tbx5; Mef2c, M₃Gata4, M₃Hand2, and M₃Tbx5; Mef2cVP16, M₃Gata4, M₃Hand2, and M₃Tbx5; Mef2cVP16, M₃Gata4, M₃Hand2, M₃Tbx5, Mef2c, Gata4, Hand2, and Tbx5; Mef2cVP16, M₃Gata4, M₃Hand2, M₃Tbx5, M₃Ets2, and M₃Mesp1; Mef2cVP16, M₃Gata4, M₃Hand2, M₃Tbx5, Mef2c, Gata4, Hand2, Tbx5, Ets2, and Mesp1; or Mef2cVP16, M₃Gata4, M₃Hand2, M₃Tbx5, M₃Ets2, M₃Mesp1, Mef2c, Gata4, Hand2, Tbx5, Ets2, and Mesp1 are introduced into the cell.

In one embodiment, the cell is mammalian, such as human. In another embodiment, the somatic cell is a fibroblast cell derived from cardiac and non-cardiac tissues and a non-fibroblast cell of any tissue origin, including keratinocyte, myocyte, satellite cell, tendon cell, chondrocyte, osteocyte, adipocyte, endothelial cell, angioblast, mesoangioblst, pericyte, mural cell, mesangial cell, juxtaglomerular cell, macula densa cell, stromal cell, epidermal cell, blood cell, bone marrow cell, and germ cell, and any types of stem cell.

One embodiment provides a fusion nucleic acid or protein comprising a heterologous transactivation domain and a transcription factor. In one embodiment, the fusion is DNA or RNA. In another embodiment, the transcription factor is Gata4, Mef2c, Tbx5, Hand2, Ets2, or Mesp1. In one embodiment the transactivation domain (TAD) is obtained from MyoD or VP16. In one embodiment, the transactivation domain (TAD) of MyoD comprises or codes for amino acids 1-62 of MyoD or is at least 80% identical thereto.

Another embodiment provides for a method comprising: (1) isolating and collecting a somatic cell from a subject; (2) inducing said somatic cell from the subject into a CM according to the method of claim 1 and (3) administering an effective amount of the CM to the subject in need thereof. In one embodiment, the somatic cell is a fibroblast cell. In another embodiment, the fibroblast cell is a non-cardiac fibroblast cell or a cardiac fibroblast cell.

### BRIEF DESCRIPTION OF THE DRAWINGS

### Abbreviations, Generic Names, and Acronyms Used in Figures

M-GHT: Mef2c, Gata4, Hand2, and Tbx5; MM₃-GHT: Mef2cM₃, Gata4, Hand2, and Tbx5; MVP16-GHT: Mef2cVP16, Gata4, Hand2, and Tbx5; M₃G-HMT: M₃Gata4, Hand2, Mef2c, and Tbx5; M₃H-GMT: M₃Hand2, Gata4, Mef2c, and Tbx5; M₃T-HMT: M₃Tbx5, Gata4, Hand2, and Mef2c; cTnT: cardiac troponin T; cMHC: cardiac myosin heavy chain; MLC2v: myosin light chain 2v; Scn1b: sodium channel, voltage-gated, type I, beta subunit; Nav1.5/Scn5a: sodium channel, voltage-gated, type V, alpha subunit; Hcn4: hyperpolarization-activated cation channel 4.

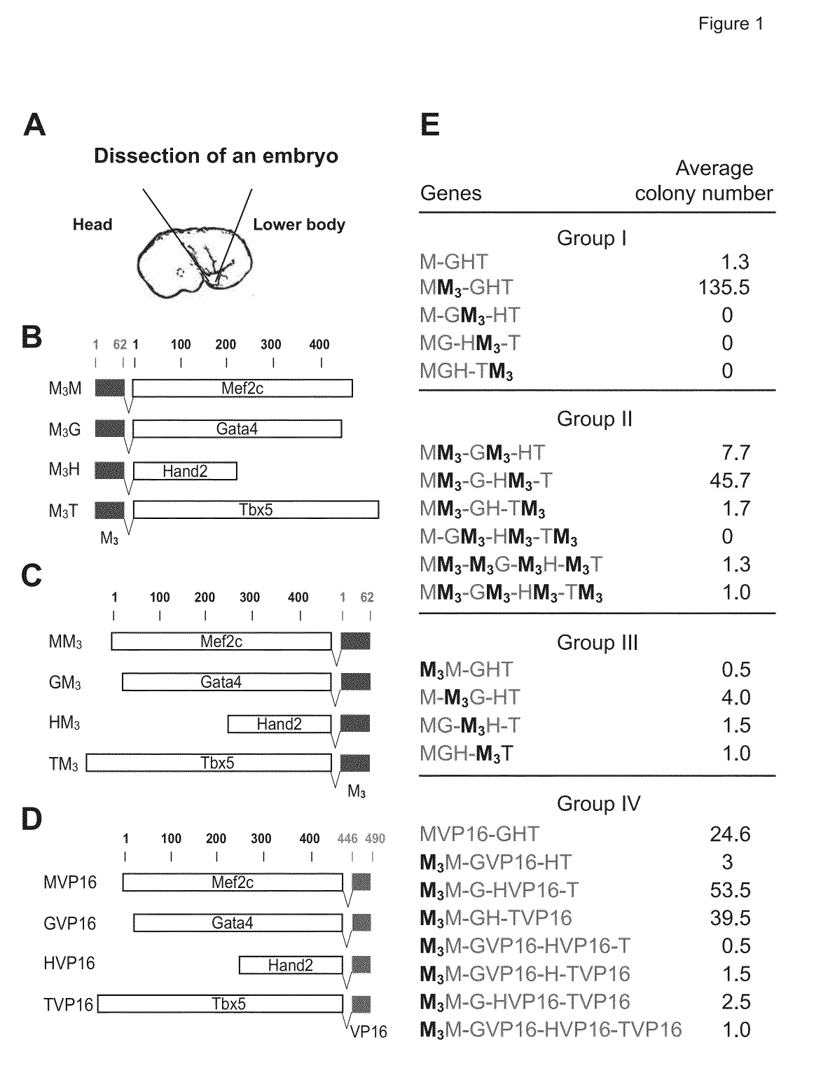

FIG. 1. Establishment of beating iCM clusters from embryonic fibroblasts with M3 domain-fusion genes. (A) Fibroblasts were separately prepared from the head and the lower body of mouse embryos at E13.5. (B) Fusion constructs of the M3 domain attached to the amino-terminus of the 4 cardiac genes used in the study. Amino acid numbers are shown at the top. (C) Fusion constructs of the M3 domain attached to the carboxy-terminus of the 4 cardiac genes. (D) Fusion constructs of the VP16 transactivation domain attached to the carboxy-terminus of the 4 cardiac genes. (E) The number of beating iCM clusters prepared from head fibroblasts with various fusion gene combinations on day 14. Fibroblasts were seeded into 48-well plates. The total number of beating iCM clusters in each well was obtained from more than 3 experiments with 2 to 3 wells in each experiment.

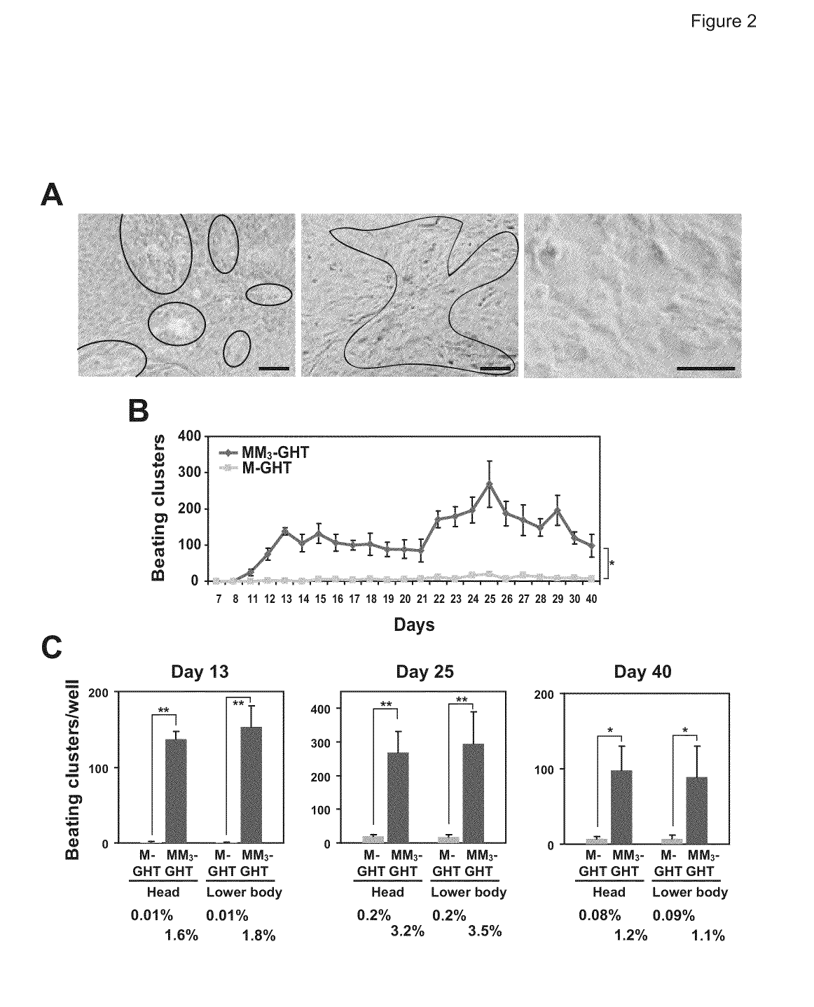

FIG. 2. Long-term culture of beating iCM clusters produced from embryonic fibroblasts. (A) Phase-contrast images of day 13 small (left) and large (middle) beating iCM clusters (encircled) prepared with Mef2cM3, Gata4, Hand2, and Tbx5 (MM3-GHT). The entire area is beating in the right panel. Bars, 100 μm. (B) The number of beating iCM clusters produced from head fibroblasts with Mef2c, Gata4, Hand2, and Tbx5 (M-GHT) and MM3-GHT over 40 days. Fibroblasts were seeded at 8,400 cells per well into 48-well plates. The number of beating iCM clusters in each well is shown as mean±SD of more than 3 experiments. Asterisk indicates an experimental pair where the difference between the compared values was statistically significant. *P<0.05 (Student's t-test). (C) The mean+SD of beating iCM clusters produced with M-GHT and MM3-GHT on days 13, 25, and 40. The reprogramming efficiency (%) is shown under each graph. Fibroblasts were seeded into 48-well plates. **P<0.01 (Student's t-test).

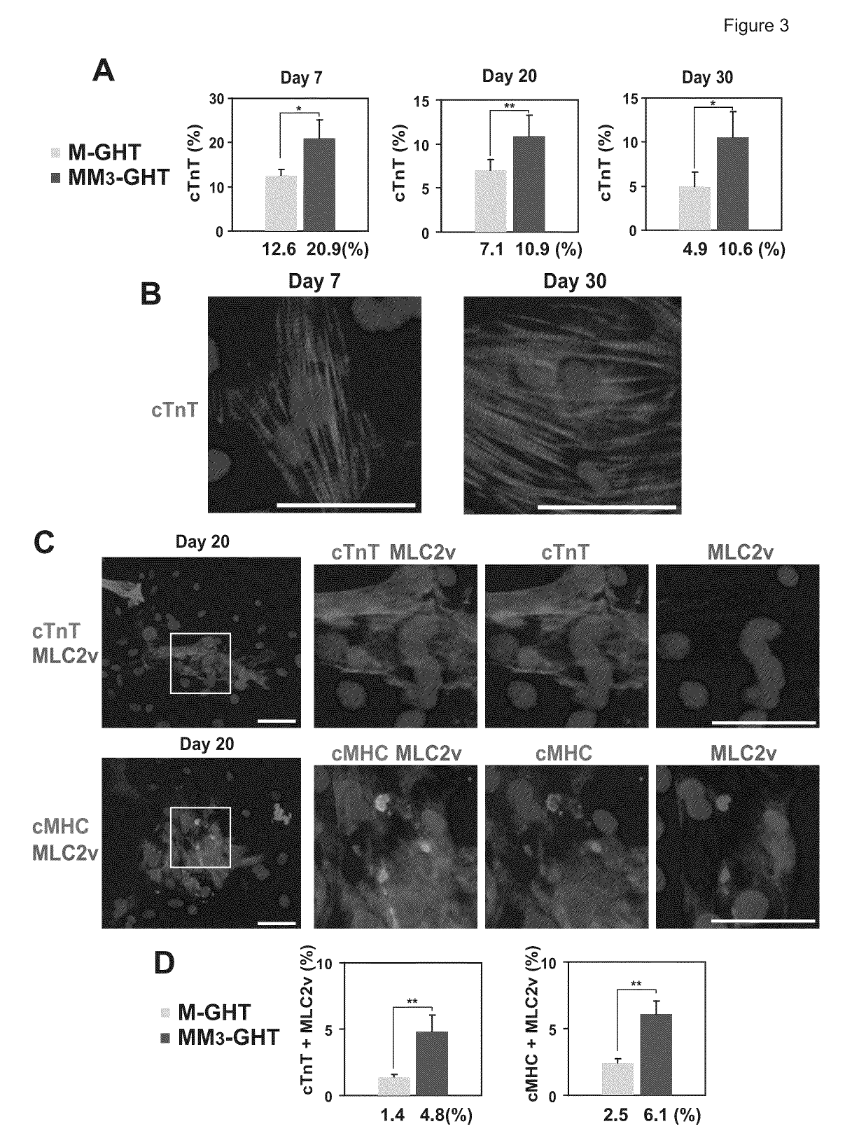

FIG. 3. Expression of cardiac cytoskeletal proteins in embryonic head fibroblasts after transduction with M-GHT or MM3-GHT. (A) Frequency of cells positive for cTnT on days 7, 20, and 30. Frequency was calculated as the number of positive cells divided by the total number of cells stained with Hoechst 33342 and is shown as mean+SD of more than 3 experiments. Asterisks indicate experimental pairs where the difference between the compared values was statistically significant. *P<0.05 and **P<0.01 (Student's t-test). (B) Immunofluorescence staining of cardiac troponin T (cTnT) on day 7 and 30 after transduction with MM3-GHT. DNA was counterstained with Hoechst 33342 (blue). Bars, 50 μm. (C) Double immunofluorescence staining of cTnT with myosin light chain 2v (MLC2v), and cardiac myosin heavy chain (cMHC) with MLC2v on day 20 after transduction with MM3-GHT. DNA was counterstained with Hoechst 33342. The areas surrounded by the rectangles in the left panels were magnified in the right three panels in each row. Bars, 50 μm for all panels. (D) Frequency of cells double-positive for cTnT and MLC2v, and cMHC and MLC2v on days 20. **P<0.01 (Student's t-test).

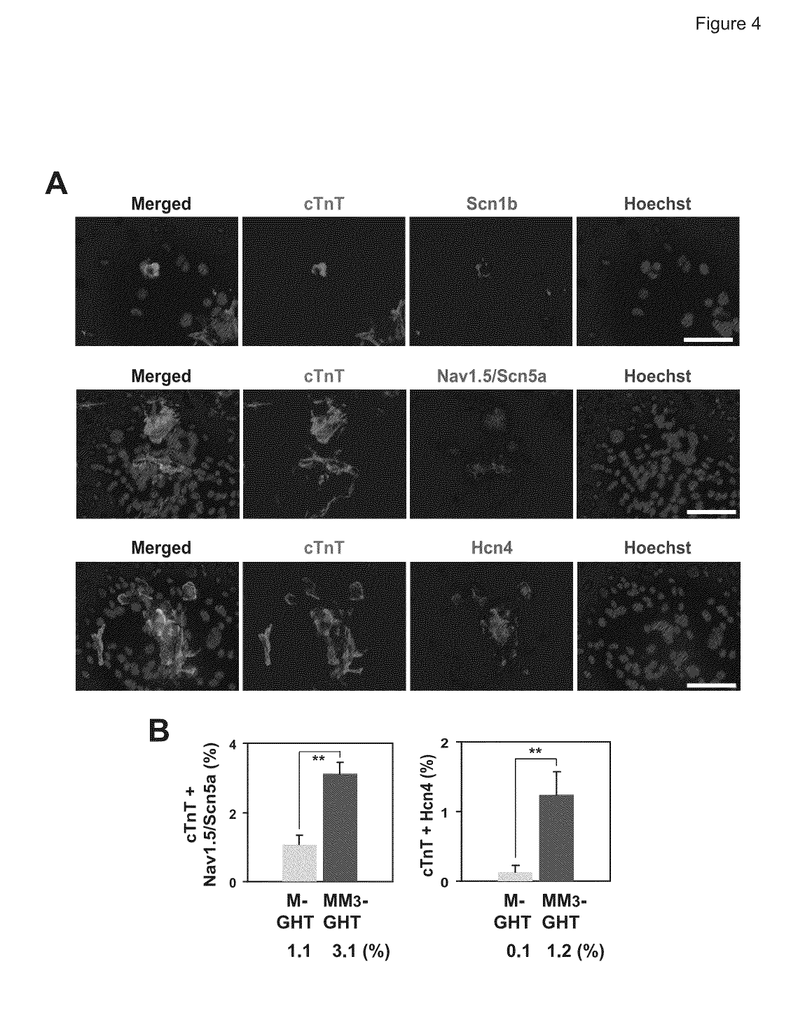

FIG. 4. Expression of ion channel proteins in iCMs generated from embryonic head fibroblasts with M-GHT or MM3-GHT. (A) Immunofluorescence staining of cardiac troponin T (cTnT), Scn1b (sodium channel, voltage-gated, type I, beta subunit), Nav1.5/Scn5a (sodium channel, voltage-gated, type V, alpha subunit), and Hcn4 (hyperpolarization-activated cation channel 4) on day 20 after transduction with MM3-GHT. DNA was counterstained with Hoechst 33342. Bars, 100 μm. (B) Frequency of cells double-positive for cTnT and Nav1.5/Scn5a, and cTnT and Hcn4 on day 20. The frequency of positive cells (%) was calculated by dividing the number of positive cells by the total number of cells stained with Hoechst and is shown as mean+SD of more than 3 experiments. Asterisks indicate experimental pairs where the difference between the compared values was statistically significant. **P<0.01 (Student's t-test).

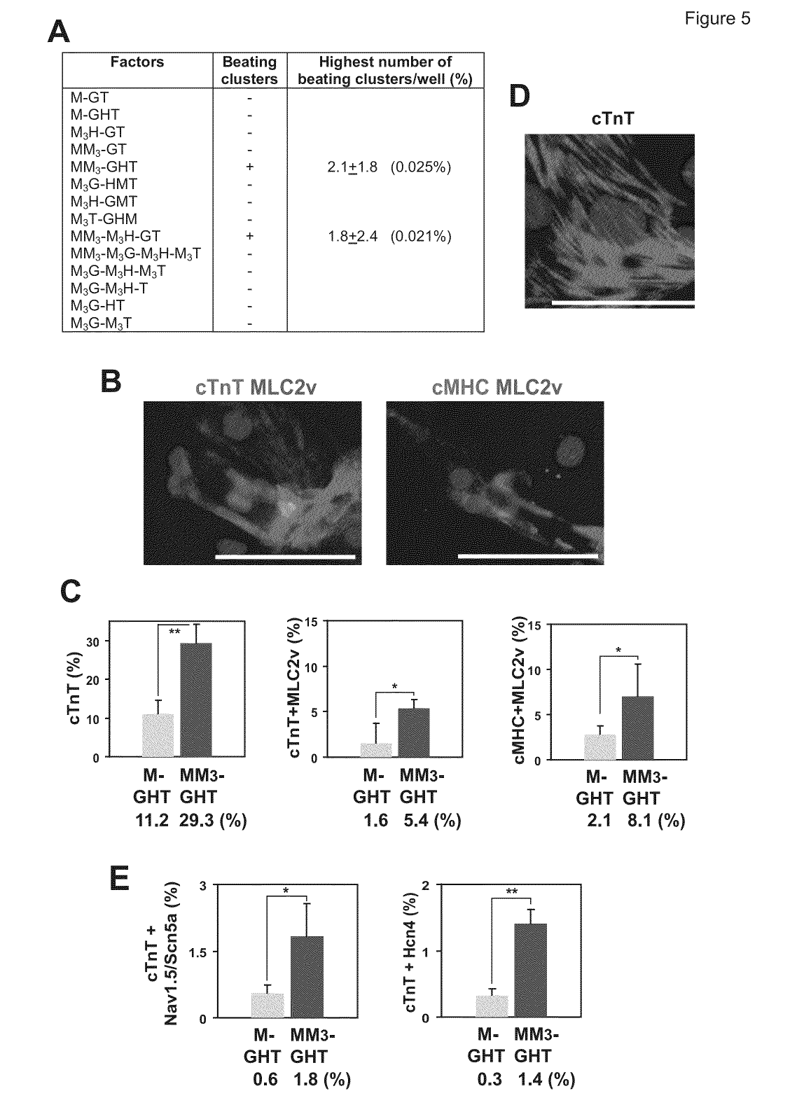

FIG. 5. Formation of beating iCMs from neonatal tail fibroblasts. (A) Comparison at day 20 of the highest number of beating iCM clusters obtained with various gene combinations. The total number of iCM clusters was counted in each well of a 48-well plate. Means±SD of more than 3 experiments and percent efficiency are shown. (B) Double immunofluorescence staining of cardiac troponin T (cTnT) and myosin light chain 2v (MLC2v), and cardiac myosin heavy chain (cMHC) and MLC2v on day 20 after transduction with MM3-GHT. DNA was counterstained with Hoechst 33342. Bars, 100 μm. (C) Frequency (%) of cells positive for cTnT alone, double-positive for cTnT and MLC2v, and cMHC and MLC2v on day 20. Asterisks indicate experimental pairs where the difference between the compared values was statistically significant. *P<0.05 and **P<0.01 (Student's t-test). (D) Sarcomere-like structures observed with cTnT staining on day 20 after transduction with MM3-GHT. DNA was counterstained with Hoechst 33342. Bar, 50 μm. (E) Frequency of cells positive for cTnT and ion channel proteins on day 30.

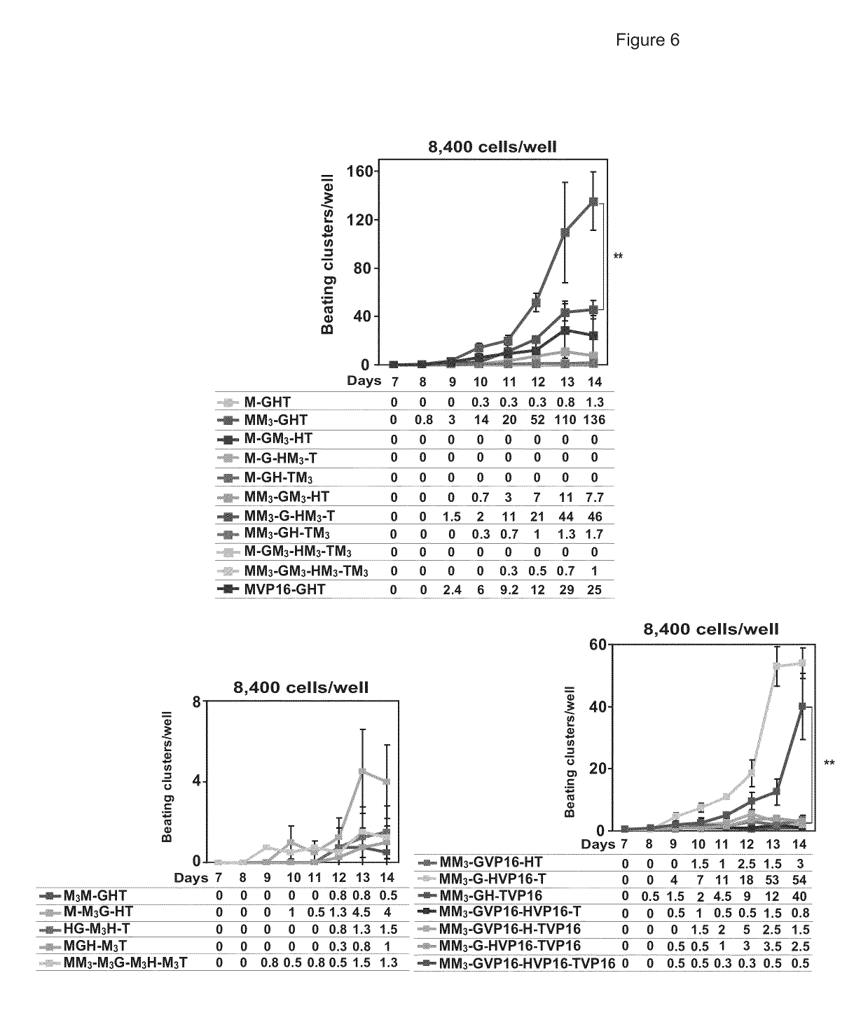

FIG. 6. The number of beating iCM clusters generated from head fibroblasts over 2 weeks. Fibroblasts were seeded into 48-well plates. The total number of beating iCM clusters in each well is shown as mean±standard deviation (SD) of more than 3 experiments. Means are also given below the graph. **P<0.01 (Student's t-test).

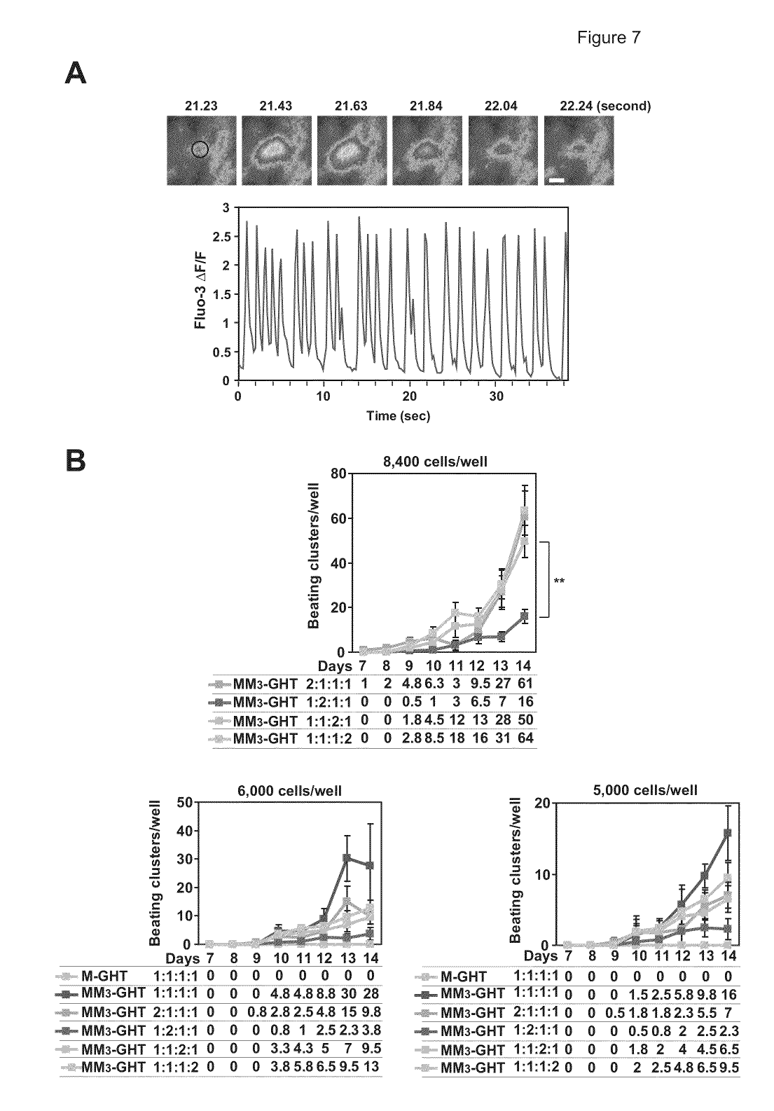

FIG. 7. Calcium transients and the number of beating iCM clusters prepared from head fibroblasts. (A) Calcium transients in beating iCM cells emerged from head fibroblasts on day 20 after transduction with MM3-GHT. Top panels show a series of fluorescence images depicting the calcium concentration in the cells. The bottom graph displays the fluctuation of the calcium concentration in the area marked by a circle in the top left panel. Bar, 10 μm. (B) The number of beating iCM clusters produced from head fibroblasts with varying gene ratios and cell densities. Fibroblasts were seeded into 48-well plates. **P<0.01 (Student's t-test).

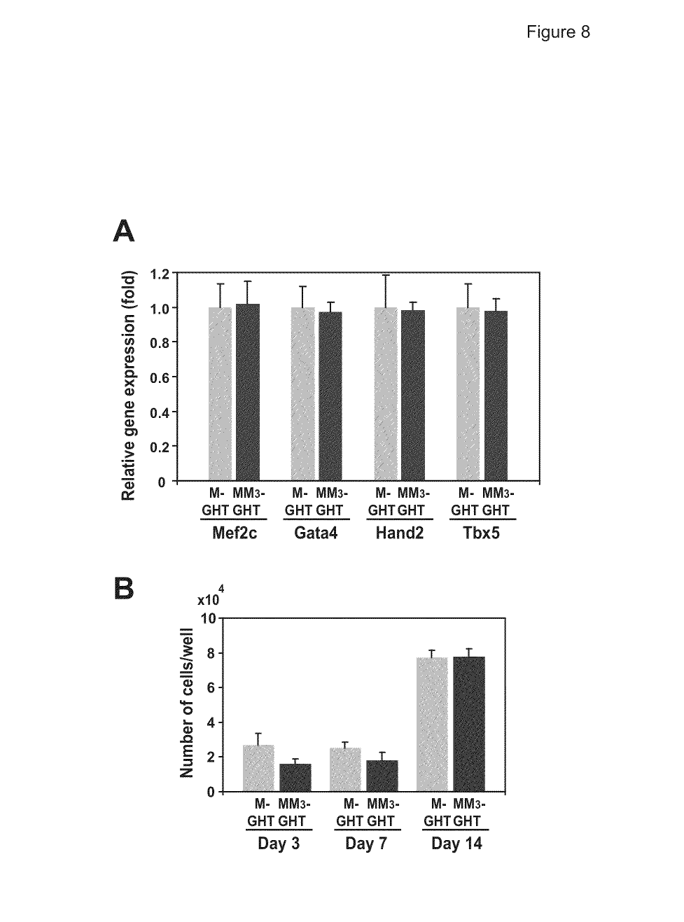

FIG. 8. The expression levels of the transduced genes and cell numbers over time. (A) Relative mRNA levels of Mef2c, Gata4, Hand2, and Tbx5 in head fibroblasts on day 1 after transduction with M-GHT and MM3-GHT. The value obtained with M-GHT was defined as 1.0 for each gene. Mean+standard deviation (SD) of more than 3 experiments is shown. (B) The number of total cells in one well of a 48-well plate of a head fibroblast culture counted on day 3, 7, and 14 after transduction with M-GHT or MM3-GHT. Mean+SD obtained from three independent experiments are displayed.

### DETAILED DESCRIPTION OF THE INVENTION

Herein is described the fusion of the M₃ domain to Mef2c (Mef2cM₃) and other cardiac genes/proteins, along with transduction of the fusion with Gata4, Hand2 and Tbx5 (Mef2cM₃-GHT) into mouse embryonic fibroblasts (MEFs). This strategy succeeded to improve the efficiency of converting MEFs into beating CMs more than tenfold (1% of transduced MEFs) in comparison to the combination between Mef2c, Gata4, Hand2 and Tbx5 (Mef2c-GHT). In addition, immunofluorescence staining showed that many of the non-beating cells prepared with Mef2cM₃-GHT expressed several proteins specifically expressed in terminally differentiated CMs, suggesting advanced partial reprogramming. Such partially reprogrammed cells were rare with Mef2c-GHT. As only certain types of CMs spontaneously beat, the partially reprogrammed CMs may be fully differentiated CMs that do not spontaneously beat. Thus, the M₃ domain does facilitate/amplify the function of the Mef2c protein (the fusion partner) or other cardiac genes/proteins.

By fusing the highly potent transactivation domain derived from MyoD (M3 domain) to Mef2c, a chimeric protein Mef2cM₃ was created. Transduction of Mef2cM₃ along with Gata4, Hand3 and Tbx5 (Mef2cM₃-GHT) substantially improved the efficiency of converting non-cardiac fibroblasts into beating CM-like cells called induced CM-like cells (iCMs). Specifically, while Mef2c with GHT (Mef2c-GHT) did not generate iCMs from mouse neonatal tail fibroblasts without the CM conditioned medium, MM₃-GHT and MM₃-M₃H-GT allowed for the generation by day 35 without the conditioned medium (FIG. 1D). In addition, when embryonic fibroblasts were used as the parent cells, Mef2cM₃-GHT formed iCMs more than 10-fold efficiently and several days earlier than Mef2c-GHT (FIGS. 3D and 3E).

### DEFINITIONS

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “isolated” refers to a factor(s), cell or cells which are not associated with one or more factors, cells or one or more cellular components that are associated with the factor(s), cell or cells in vivo.

“Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, or orangutan), rat, sheep, goat, cow and bird.

An “effective amount” generally means an amount which provides the desired local or systemic effect and/or performance.

“Pluripotency” refers to a stem cell that has the potential to differentiate into one, two or three of the three germ layers: endoderm (e.g., interior stomach lining, gastrointestinal tract, the lungs), mesoderm (e.g., muscle, bone, blood, urogenital), or ectoderm (e.g., epidermal tissues and nervous system). Pluripotent stem cells can give rise to any fetal or adult cell type.

“Transdifferentiation” is when a non-stem cell transforms into a different type of cell, or when an already differentiated stem cell creates cells outside its already established differentiation path.

A “transcription factor” (sometimes called a sequence-specific DNA-binding factor) is a protein that binds to specific DNA sequences, thereby controlling the transfer (or transcription) of genetic information from DNA to mRNA. Transcription factors perform this function alone or with other proteins or factors in a complex, by promoting (as an activator), or blocking (as a repressor) the recruitment of RNA polymerase (the enzyme that performs the transcription of genetic information from DNA to RNA) to specific genes. Generally, a defining feature of transcription factors is that they contain one or more DNA-binding domains (DBDs), which attach to specific sequences of DNA adjacent to the genes that they regulate.

A “transcription activation domain,” “transactivation domain” or “trans-activating domain” is generally that portion of a transcription factor that is responsible for recruitment of the transcription machinery needed to transcribe RNA. Transactivation is an increased rate of gene expression triggered either by biological processes or by artificial means. Transactivation can be triggered either by endogenous cellular or viral proteins—transactivators. These protein factors can act in trans (i.e., intermolecularly). An “unrelated” or “heterologous transactivation domain” refers to a transactivation domain that is not normally associated with the gene/protein (e.g., transcription factor) of interest (not wild-type).

By “pure” it is meant that the population of cells has the desired purity. For example, CM populations can comprise mixed populations of cells. Those skilled in the art can readily determine the percentage of CMs in a population using various well-known methods, such as fluorescence activated cell sorting (FACS). Preferable ranges of purity in populations comprising CMs or other cells are about 1 to about 5%, about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90% to about 95% or about 95 to about 100%. Purity of the cells can be determined for example according to the cell surface marker profile within a population.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy or amino terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.
As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

As used herein, an “effective amount” means an amount sufficient to produce a selected effect.

The term “inhibit,” as used herein, refers to the ability of a compound or any agent/composition to reduce or impede a described function, level, activity, synthesis, release, binding, etc., based on the context in which the term “inhibit” is used. For example, inhibition is by at least about 10%, such as by at least about 25%, including by at least about 50%, or wherein the function is inhibited by at least about 75%. The term “inhibit” is used interchangeably with “reduce” and “block.”

As used herein, the term “nucleic acid” encompasses RNA as well as single, double and triple stranded DNA and cDNA. Furthermore, the terms, “nucleic acid,” “DNA,” “RNA” and similar terms also include nucleic acid analogs, i.e. analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is also meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “small interfering RNAs (siRNAs)” is meant, inter alia, an isolated dsRNA molecule comprising both a sense and an anti-sense strand. In one aspect, it is greater than 10 nucleotides in length. siRNA also refers to a single transcript that has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using, for example, the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, and which is not deleterious to the subject to which the composition is to be administered. In many cases, the compounds of the present invention are capable of forming acid and/or base salts by virtue of the presence of amino and/or carboxyl groups or groups similar thereto.

The term “prevent,” as used herein, means to stop or inhibit something from happening, or taking advance measures against something possible or probable from happening. In the context of medicine, “prevention” generally refers to action taken to decrease the chance of getting a disease or condition.

As used herein, the term “purified” and like terms relate to an enrichment of a molecule, compound or cell relative to other components normally associated with the molecule or compound in a native environment. The term “purified” does not necessarily indicate that complete purity of the particular molecule has been achieved during the process. A “highly purified” compound/cell population as used herein refers to a compound that is greater than 90% pure.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, an “instructional material” for use in a kit, for example, includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The terms “comprises,” “comprising,” and the like can have the meaning ascribed to them in U.S. Patent Law and can mean “includes,” “including” and the like. As used herein, “including” or “includes” or the like means including, without limitation.

### Rapid and Efficient Production of CMs

Methods of Generating iCMs

Nucleic acid or protein constructs can be introduced into a host cell using a variety of techniques, such as non-viral or viral transfection of the cell. In one embodiment, the construct is directly introduced into a host cell or incorporated into a vector and introduced into a host cell, or incorporated into a virus vector and introduced into a host cell as virus. Examples of the constructs include, but are not limited to, nucleic acids, such as DNA or RNA, or protein, vectors, such as plasmids, bacterial artificial chromosomes, yeast artificial chromosomes, human artificial chromosomes, cDNA, cRNA, and polymerase chain reaction (PCR) product expression cassettes and recombinant viruses.

Introduction into the cell may be performed by any non-viral or viral transfection available in the art, such as, but not limited to, electroporation, microinjection, cell fusion, heat shock, magnetofection, microprojectile-mediated transfer (nanoparticles), calcium phosphate-mediated transfer, nucleofection, liposome-mediated transfer, cationic polymer-mediated transfer, any physical-, chemical-, and biological-transfer, sendaivirus, retrovirus, lentivirus, adenovirus, and any other viruses. Other methods of transfection include transfection reagents such as Lipofectamine™, Fugene™, Transfectamine™, etc.

### Reprogramming Factors

Through the processes disclosed herein, beating iCMs emerge as early as about seven or eight days through transduction of a transactivator domain (or a portion thereof) fused to a transcription factor (or a portion thereof), e.g., M₃ (transactivation domain of MyoD (about 50 to 60 amino acids) fused to the amino terminus of the full-length (or partial length) of Mef2c, Gata4, Hand2, Tbx5, Ets2, and/or Mesp1 without feeder cells. The preparation of the nucleic acid molecule coding for the fusion protein(s) as well as the construct(s) of. (either singly or on a polycistronic RNA) can be carried out by methods available to an art worker as well as the transduction thereof into cells (see, for example, Sambrook, Molecular Cloning: A Laboratory Manual).

Generally, genes/nucleotide sequences encoding one or more reprogramming factors which can be used to create iCMs from fibroblasts or other non-cardiac cells, either singly, in combination or as fusions with transactivation domains, include, but are not limited to, one or more of the following: Mef2cM₃, Mef2cVP16, M₃Gata4, M₃Hand2, M₃Tbx5, M₃Ets2, M₃Mesp1, Mef2c, Gata4, Hand2, Tbx5, Ets2, and Mesp1.

In some embodiments, the one or more reprogramming factors include(s) the following combinations 1, 2, 3, 4, 5, 6, 7, 8, 9, 10, 11, 12, and/or 13 of Mef2cM₃, Mef2cVP16, M₃Gata4, M₃Hand2, M₃Tbx5, M₃Ets2, M₃Mesp1, Mef2c, Gata4, Hand2, Tbx5, Ets2, and/or Mesp1.

Exemplary subsets include: Mef2cM₃, Gata4, Hand2, and Tbx5; Mef2c, M₃Gata4, Hand2, and Tbx5; Mef2c, Gata4, M₃Hand2, and Tbx5; Mef2c, Gata4, Hand2, and M₃Tbx5; Mef2cM₃, M₃Gata4, Hand2, and Tbx5; Mef2cM₃, Gata4, M₃Hand2, and Tbx5; Mef2cM₃, Gata4, Hand2, and M₃Tbx5; Mef2c, M₃Gata4, M₃Hand2, and Tbx5; Mef2c, M₃Gata4, Hand2, and M₃Tbx5; Mef2c, Gata4, M₃Hand2, and M₃Tbx5; Mef2cM₃, M₃Gata4, M₃Hand2, and Tbx5; Mef2cM₃, M₃Gata4, Hand2, and M₃Tbx5; Mef2cM₃, Gata4, M₃Hand2, and M₃Tbx5; Mef2c, M₃Gata4, M₃Hand2, and M₃Tbx5; Mef2cM₃, M₃Gata4, M₃Hand2, and M₃Tbx5; Mef2cM₃, M₃Gata4, M₃Hand2, M₃Tbx5, Mef2c, Gata4, Hand2, and Tbx5; Mef2cM₃, M₃Gata4, M₃Hand2, M₃Tbx5, M₃Ets2, and M₃Mesp1; Mef2cM₃, M₃Gata4, M₃Hand2, M₃Tbx5, Mef2c, Gata4, Hand2, Tbx5, Ets2, and Mesp1; and Mef2cM₃, Mef2cVP16, M₃Gata4, M₃Hand2, M₃Tbx5, M₃Ets2, M₃Mesp1, Mef2c, Gata4, Hand2, Tbx5, Ets2, and Mesp1.

### Nucleotide Sequences of the Reprogramming Factors

1) MyoD TAD

Nucleic acids encoding the M₃ domain of MyoD (amino acids 1-62)

### Mouse MyoD: M84918, NM_{—}010866

### Human MyoD: NM_{—}002478

2) VP16

Nucleic acids encoding the VP16 (amino acids 411-490, 446-490)
Human herpesvirus 1 complete genome: X14112.1
Tegument protein VP16 from human herpes simplex virus type 1: NP_{—}044650

3) Mef2c

Myocyte-specific enhancer factor 2c (Mef2c) is a transcription factor that recognizes and binds to the MEF2 element present in the regulatory regions of many muscle-specific genes, and activates transcription of the genes linked to such promoters.
Mouse Mef2c: NM_{—}001170537, NM_{—}025282
Human Mef2c: BC156603, NM_{—}001131005, NM_{—}001193347, NM_{—}001193348, NM_{—}001193349, NM_{—}001193350, NM_{—}002397
Rattus norvegicus: Mef2c: XM_{—}003749164
Macaca mulatta Mef2c: NM_{—}001266447, XM_{—}001086519, XM_{—}001086651, XM_{—}001087589

4) Gata4

Gata4 is a member of the GATA family zinc-finger transcription factor that recognizes and binds to a GATA motif present in the promoter region of many genes, and activates transcription of the genes linked to such promoters.

### Mouse Gata4: NM_{—}008092

### Human Gata4:NM_{—}002052

Rattus norvegicus Gata4:NM_{—}144730
Macaca mulatta Gata4: XM_{—}001087008

5) Hand2

Heart- and neural crest derivatives-expressed protein 2 (Hand2) is a transcription factor that recognizes and binds to the E-box consensus sequence 5′-CANNTG-3′, and activates transcription of genes linked to such promoters.
Mouse Hand2: NM 010402
Human Hand2: NM_{—}021973
Rattus norvegicus Hand2: NM_{—}022696
Macaca mulatta Hand2: NM_{—}001193765, XM_{—}001085842, XM_{—}001085733

6) Tbx5

T-box transcription factor 5 (Tbx5) is a transcription factor that recognizes and binds specifically to the T-box binding element consensus sequence 5′-(A/G)GGTGT(C/G/T)(A/G)-3′ in the promoter region of many genes, and activates transcription of the genes linked to such promoters.
Mouse Tbx5: NM_{—}011537
Human Tbx5: NM_{—}000192, NM_{—}080717, NM_{—}181486
Rattus norvegicus Tbx5: NM_{—}001009964
Macaca mulatta Tbx5: XM_{—}001111777, XM_{—}001111737

7) Ets2

V-ets erythroblastosis virus E26 oncogene homolog 2 (Ets2) is a transcription factor that regulates genes involved in development and apoptosis.
Mouse Ets2: NM_{—}011809
Human Ets2: NM_{—}001256295, NM_{—}005239
Rattus norvegicus Ets2: NM_{—}001107107
Macaca mulatta Ets2: XM_{—}001109324

8) Mesp1

Mesoderm posterior 1 homolog (Mesp1) is a basic helix-loop-helix-type transcription factor expressed in the precursors of the cardiovascular cells and is known to play a role in cardiac morphogenesis.
Mouse Mesp1: NM_{—}008588
Human Mesp1: NM_{—}018670
Rattus norvegicus Mesp1: NM_{—}001107531
Macaca mulatta Mesp1: XM_{—}001093487

or portions or fragments thereof and/or any related sequence available to an art worker (these sequences are incorporated by referenced herein). For example, sequences for use in the invention have at least about 50% or about 60% or about 70%, about 71%, about 72%, about 73%, about 74%, about 75%, about 76%, about 77%, about 78%, or about 79%, or at least about 80%, about 81%, about 82%, about 83%, about 84%, about 85%, about 86%, about 87%, about 88%, or about 89%, or at least about 90%, about 91%, about 92%, about 93%, or about 94%, or at least about 95%, about 96%, about 97%, about 98%, or about 99% sequence identity compared to the sequences and/or accession numbers provided herein and/or any other such sequence available to an art worker, using one of alignment programs available in the art using standard parameters or hybridization techniques. In one embodiment, the differences in sequence are due to conservative amino acid changes. In another embodiment, the protein sequence or DNA sequence has at least 80% sequence identity with the sequences disclosed herein and is bioactive (e.g., retains activity).

### Growth/Maintenance of Cells

During and after preparation of the fibroblasts/iCMs, the cells can be cultured in culture medium that is established in the art and commercially available from the American Type Culture Collection (ATCC), Invitrogen and other companies. Such media include, but are not limited to, Dulbecco's Modified Eagle's Medium (DMEM), DMEM F12 medium, Eagle's Minimum Essential Medium, F-12K medium, Iscove's Modified Dulbecco's Medium, Knockout D-MEM, or RPMI-1640 medium. It is within the skill of one in the art to modify or modulate concentrations of media and/or media supplements as needed for the cells used. It will also be apparent that many media are available as low-glucose formulations, with or without sodium pyruvate.

Also contemplated is supplementation of cell culture medium with mammalian sera. Sera often contain cellular factors and components that are needed for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, rat serum (RS), serum replacements (including, but not limited to, KnockOut Serum Replacement (KSR, Invitrogen)), and bovine embryonic fluid. It is understood that sera can be heat-inactivated at 55-65° C. if deemed needed to inactivate components of the complement cascade. Modulation of serum concentrations, or withdrawal of serum from the culture medium can also be used to promote survival of one or more desired cell types. In one embodiment, the cells are cultured in the presence of FBS/or serum specific for the species cell type. For example, cells can be isolated and/or expanded with total serum (e.g., FBS) or serum replacement concentrations of about 0.5% to about 5% or greater including about 5% to about 15% or greater, such as about 20%, about 25% or about 30%. Concentrations of serum can be determined empirically.

Additional supplements can also be used to supply the cells with trace elements for optimal growth and expansion. Such supplements include insulin, transferrin, sodium selenium, and combinations thereof. These components can be included in a salt solution such as, but not limited to, Hanks' Balanced Salt Solution™ (HBSS), Earle's Salt Solution™, antioxidant supplements, MCDB-201™ supplements, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids. Many cell culture media already contain amino acids; however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Antibiotics are also typically used in cell culture to mitigate bacterial, mycoplasmal, and fungal contamination. Typically, antibiotics or anti-mycotic compounds used are mixtures of penicillin/streptomycin, but can also include, but are not limited to, amphotericin (Fungizone™) ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. β-mercaptoethanol can also be supplemented in cell culture media.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulation.

Cells in culture can be maintained either in suspension or attached to a solid support, such as extracellular matrix components and synthetic or biopolymers. Cells often require additional factors that encourage their attachment to a solid support (e.g., attachment factors) such as type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin.

Cells can be cultured at different densities, e.g., cells can be seeded or maintained in the culture dish at different densities. For example, at densities, including, but not limited to, densities of less than about 2000 cells/well of a 12-well plate (for example, 12-well flat-bottom growth area: 3.8 cm2 well volume: 6.0 ml or well ID×depth (mm) 22.1×17.5; well capacity (ml) 6.5, growth area (cm2) 3.8), including less than about 1500 cells/well of a 12-well plate, less than about 1,000 cells/well of a 12-well plate, less than about 500 cells/well of a 12-well plate, or less than about 200 cells/well of a 12-well plate. The cells can also be seeded or maintained at higher densities, for example, great than about 2,000 cells/well of a 12-well plate, greater than about 2,500 cells/well of a 12-well plate, greater than about 3,000 cells/well of a 12-well plate, greater than about 3,500 cells/well of a 12-well plate, greater than about 4,000 cells/well of a 12-well plate, greater than about 4,500 cells/well of a 12-well plate, greater than about 5,000 cells/well of a 12-well plate, greater than about 5,500 cells/well of a 12-well plate, greater than about 6,000 cells/well of a 12-well plate, greater than about 6,500 cells/well of a 12-well plate, greater than about 7,000 cells/well of a 12-well plate, greater than about 7,500 cells/well of a 12-well plate or greater than about 8,000 cells/well of a 12-well plate.

### Therapeutic Uses

The iCMs described herein have many clinical and preclinical (e.g., research) applications. For example, the cells described herein can be used for tissue repair (such as for repair of damaged heart tissue, such as damage caused by disease (e.g., myocardial infarction) or injury (e.g., surgical or accident).

Matrices can be used to deliver cells of the present invention to specific anatomic sites, where particular growth factors can be incorporated into the matrix, or encoded on plasmids incorporated into the matrix for uptake by the cells, can be used to direct the growth of the initial cell population. DNA can be incorporated within pores of the matrix, for example, during the process used in the formation of certain polymer matrices. As the polymer used in the foaming process expands, it entraps the DNA within the pores, allowing controlled and sustained release of plasmid DNA.

Plasmid DNA encoding cytokines, growth factors, or hormones can be trapped within a polymer gene-activated matrix carrier. The biodegradable polymer is then implanted near the heart, where iCMs are implanted and take up the DNA, which causes the iCMs to produce a high local concentration of the cytokine, growth factor, or hormone, accelerating healing of the damaged tissue.

For the purposes described herein, either autologous or allogeneic iCMs of the present invention can be administered to a patient by direct injection to a heart site, systemically, on or around the surface of an acceptable matrix, or in combination with a pharmaceutically acceptable carrier.

Additionally, nucleic acid constructs or proteins can be injected locally or systemically into a subject, with, for example, a pharmaceutically acceptable carrier.

### EXAMPLES

The following examples are provided in order to demonstrate and further illustrate certain embodiments and aspects of the present invention and are not to be construed as limiting the scope thereof.

### Example 1

### Preparation of Mouse Fibroblasts

Mouse embryonic fibroblasts (MEFs) were isolated from day 13.5 embryos under a dissection microscope (Leica). Following removal of all the internal organs, embryos were dissected into three parts: head, upper body, and lower body (FIG. 2A). The three parts were separately sliced into small pieces and incubated with Collagenase/Dispase (Roche Diagnostics) for 10-15 minutes to prepare a single cell suspension. The cells from each embryo were plated onto a 10 cm tissue culture dish in fibroblast medium containing Dulbecco's Modified Eagle Medium (DMEM) with 10% fetal bovine serum (Hyclone), 100 U/ml penicillin and 100 μg/ml streptomycin. Cells were cultured at 37° C. for 1 to 2 days until they became confluent and then frozen for storage. After thawing, the cells were plated in the fibroblast medium for virus transduction.

Tail fibroblasts were prepared from tails of newborn mice using surgical scissors. The tails were rinsed in ethanol, washed with PBS, and then dissociated using scissors and Collagenase/Dispase. The medium was changed every 2 days. Fibroblasts migrated out of the tail fragments after 2 to 3 days. The fibroblasts were cultured in the fibroblast medium until they became confluent and then frozen for later use.

Preparation of iCMs

The M₃ domain of mouse MyoD (amino acids 1-62) was separately fused to the full length cDNAs encoding mouse Mef2c, Gata4, Hand2, and Tbx5 at the amino- or carboxy-terminus. They constructs are herein named M₃Mef2c, M₃Gata4, M₃Hand2, M₃Tbx5, Mef2cM₃, Gata4M₃, Hand2M₃, and Tbx5M₃, respectively. The second half of the transactivation domain of VP16 (amino acids 446-490) was separately fused to the carboxy-terminus of the four genes to form Mef2cVP16, Gata4VP16, Hand2VP16, and Tbx5VP16. pMXs-IP vectors encoding M₃Mef2c, M₃Gata4, M₃Hand2, M₃Tbx5, Mef2cM₃, Gata4M₃, Hand2M₃, Tbx5M₃, Mef2cVP16, Gata4VP16, Hand2VP16, Tbx5VP16, Mef2c, Gata4, Hand2, and Tbx5 were separately transfected into Plat-E cells (Cell Biolabs) with Lipofectamin 2000 (Invitrogen). The supernatant containing retrovirus encoding these genes was harvested 48 and 72 hrs later (day −1 and 0, respectively), filtered through a 0.45 μm syringe filter and transduced into fibroblasts. On day −2, 8.4×10³ fibroblasts were plated in each well of a 48-well plate in the fibroblast medium. Fresh virus supernatant was added to the fibroblasts on day −1 and day 0 with 10 μg/ml polybrene. On day 1 virus supernatant was changed to the fibroblast medium, which was replaced every other day thereafter. Beating cells appeared around day 8 and most of them developed into beating clusters and continued beating for at least 2 months.

Construct/Fusion Preparation

Constructs/Fusions can be prepared by any method available to the art, such as those disclosed in PCT/US2011/044995 (WO 2012/012708), which application and publication is incorporated herein by reference.

Following provides details of plasmid construct preparation used in the above work.

### 1) Mouse M₃Mef2c

The M₃ domain of the mouse MyoD cDNA was fused to the amino terminus of the full-length mouse Mef2c cDNA using PCR.

### PCR for Mouse M₃Mef2c

The cDNA encoding the M₃ domain of mouse MyoD (amino acids 1-62) was amplified with the primer pair, M₃F1 (CGAGGATCCGCCATGGAGCTTCTATCGCCGCCAC; SEQ ID NO: 1) and M₃Mef2cR1 (CTTTTTTCTCCCCATGTGCTCCTCCGGTTTCAG; SEQ ID NO: 2). Full length Mef2c cDNA was amplified with the primer pair, M₃Mef2cF1 (CTGAAACCGGAGGAGCACATGGGGAGAAAAAAG; SEQ ID NO: 3) and Mef2cR2 (CGCTCGAGTCTCATGTTGCCCATCCTTCAG; SEQ ID NO: 4). These PCR products were used as templates for the next PCR with the primer pair, M₃F1 and Mef2cR2. M₃Mef2c was directly subcloned between the BamHI and XhoI sites of the pMXs-IP vector.

### PCR Parameters

| | |
|---|---|
| Denature | 94° C. 2 min |
| Denature | 94° C. 15 sec* |
| Anneal | 56° C. 30 sec* |
| Extend | 68° C. 1 min* |
| Final extension | 68° C. 7 min |

*Repeat 22 cycles

### The DNA Sequence of Mouse M₃Mef2c (M₃ Domain is Underlined.)

(SEQ ID NO: 47)
atggagcttctatcgccgccactccgggacatagacttgacaggccccga
cggctctctctgctcctttgagacagcagacgacttctatgatgatccgt
gtttcgactcaccagacctgcgcttttttgaggacctggacccgcgcctg
gtgcacgtgggagccctcctgaaaccggaggagcacatggggagaaaaaa
gattcagattacgaggataatggatgagcgtaacagacaggtgactttta
cgaagaggaaatttggattgatgaagaaggcttatgagctgagcgtgctg
tgcgactgtgagattgcactgatcatcttcaacagcaccaacaagctgtt
ccagtacgccagcactgacatggataaggtgttgctcaagtacaccgagt
acaacgagccgcacgagagccggacaaactcagacattgtggagacattg
agaaagaagggcctcaatggctgtgacagcccagatcccgatgcagacga
ttcagtaggtcacagccctgagtctgaggacaagtacaggaaaattaacg
aagatattgatctaatgatcagcaggcaaagattgtgtgctgttccacct
cccagctttgagatgccagttaccatcccagtgtccagccataacagttt
ggtgtacagcaatcctgtcagcacactgggaaaccccaatcttctgccac
tggcccacccgtctctgcagaggaatagtatgtctcctggtgtaacacat
agacctccaagtgcaggtaacacaggcggtctgatgggcggagatctgac
atccggtgcaggcaccagcgcagggaatggatacggcaacccccggaact
caccaggcctgctggtctcacctggtaacctgaacaagaatatacaagcc
aaatctcctccccctatgaatctaggaatgaataatcgtaagccagatct
ccgcgttcttatcccacctggcagcaagaacacgatgccatcagtgaatc
aaaggataaataactcccagtcggctcagtcattggctaccccggtggtt
tccgtagcaactcctactttaccaggacaaggaatgggaggatatccatc
agccatttcaacaacatatggtactgagtactctctgagtagcgcagatc
tgtcatctctgtctggcttcaacactgccagtgcgctccacctcggctct
gtaactggctggcagcagcagcacctacataacatgccgccatctgccct
cagtcagttgggagaccgtaccaccaccccttcgagatacccacaacaca
ccacgcgccacgaggcggggaggtctcctgttgacagcttgagcagctgt
agcagttcctacgatgggagcgaccgagaggatcaccggaacgaattcca
ctcccccattggactcaccagaccttcgccggacgaaagggaaagtcctt
cagtcaagcgcatgcgactctctgaaggatgggcaacatga

### 2) Human M₃Mef2c

The M₃ domain of the human MyoD cDNA was fused to the amino terminus of the full-length human Mef2c cDNA using PCR.

### PCR for Human M₃Mef2c

The cDNA encoding the M₃ domain of human MyoD (amino acids 1-62) was amplified with the primer pair, hM₃F5 (CGAGGATCCATGGAGCTACTGTCGCCAC; SEQ ID NO: 5) and hM₃MEF2CR1 (GAATCTTTTTTCTCCCCATGTGCTCTTCGGGTTTCAG; SEQ ID NO: 6). Full length Mef2c cDNA was amplified with the primer pair, hM₃MEF2CF1 (CTGAAACCCGAAGAGCACATGGGGAGAAAAAAGATTC; SEQ ID NO: 7) and hMEF2CR1 (CGACTCGAGTTATGTTGCCCATCCTTC; SEQ ID NO: 8). These PCR products were used as templates for the next PCR with the primer pair, hM₃F5 and hMEF2CR1. M₃Mef2c was directly subcloned between the BamHI and XhoI sites of the pMXs-IP vector.

### PCR Parameters

| | |
|---|---|
| Denature | 94° C. 2 min |
| Denature | 94° C. 15 sec* |
| Anneal | 56° C. 30 sec* |
| Extend | 68° C. 1 min* |
| Final extension | 68° C. 7 min |

*Repeat 22 cycles

### The DNA Sequence of Human M₃Mef2c (M₃ Domain is Underlined.)

(SEQ ID NO: 48)
atggagctactgtcgccaccgctccgcgacgtagacctgacggcccccga
cggctctctctgctcctttgccacaacggacgacttctatgacgacccgt
gtttcgactccccggacctgcgcttcttcgaagacctggacccgcgcctg
atgcacgtgggcgcgctcctgaaacccgaagagcacatggggagaaaaaa
gattcagattacgaggattatggatgaacgtaacagacaggtgacattta
caaagaggaaatttgggttgatgaagaaggcttatgagctgagcgtgctg
tgtgactgtgagattgcgctgatcatcttcaacagcaccaacaagctgtt
ccagtatgccagcaccgacatggacaaagtgcttctcaagtacacggagt
acaacgagccgcatgagagccggacaaactcagacatcgtggagacgttg
agaaagaagggccttaatggctgtgacagcccagaccccgatgcggacga
ttccgtaggtcacagccctgagtctgaggacaagtacaggaaaattaacg
aagatattgatctaatgatcagcaggcaaagattgtgtgctgttccacct
cccaacttcgagatgccagtctccatcccagtgtccagccacaacagttt
ggtgtacagcaaccctgtcagctcactgggaaaccccaacctattgccac
tggctcacccttctctgcagaggaatagtatgtctcctggtgtaacacat
cgacctccaagtgcaggtaacacaggtggtctgatgggtggagacctcac
gtctggtgcaggcaccagtgcagggaacgggtatggcaatccccgaaact
caccaggtctgctggtctcacctggtaacttgaacaagaatatgcaagca
aaatctcctcccccaatgaatttaggaatgaataaccgtaaaccagatct
ccgagttcttattccaccaggcagcaagaatacgatgccatcagtgtctg
aggatgtcgacctgcttttgaatcaaaggataaataactcccagtcggct
cagtcattggctaccccagtggtttccgtagcaactcctactttaccagg
acaaggaatgggaggatatccatcagccatttcaacaacatatggtaccg
agtactctctgagtagtgcagacctgtcatctctgtctgggtttaacacc
gccagcgctcttcaccttggttcagtaactggctggcaacagcaacacct
acataacatgccaccatctgccctcagtcagttgggagcttgcactagca
ctcatttatctcagagttcaaatctctccctgccttctactcaaagcctc
aacatcaagtcagaacctgtttctcctcctagagaccgtaccaccacccc
ttcgagatacccacaacacacgcgccacgaggcggggagatctcctgttg
acagcttgagcagctgtagcagttcgtacgacgggagcgaccgagaggat
caccggaacgaattccactcccccattggactcaccagaccttcgccgga
cgaaagggaaagtccctcagtcaagcgcatgcgactttctgaaggatggg
caacataa

3) Mouse Mef2cM₃

The M₃ domain of the mouse MyoD cDNA was fused to the carboxy terminus of the full-length mouse Mef2c cDNA using PCR.

PCR for Mouse Mef2cM₃

The cDNA encoding the M₃ domain of mouse MyoD (amino acids 1-62) was amplified with the primer pair, Mef2cM₃F1 (CTGAAGGATGGGCAACAATGGAGCTTCTATCGCCG; SEQ ID NO: 9) and Mef2cM₃R2 (CGACTCGAGGAATTCTCAGTGCTCCTCCGGTTTCAG; SEQ ID NO: 10). Full length Mef2c cDNA was amplified with the primer pair, Mef2cF3 (GAGGATCCGCCATGGGGAGAAAAAAG; SEQ ID NO: 11) and Mef2cM₃R1 (CGGCGATAGAAGCTCCATTGTTGCCCATCCTTCAG; SEQ ID NO: 12). These PCR products were used as templates for the next PCR with the primer pair, Mef2cF3 and Mef2cM₃R2. Mef2cM₃ was directly subcloned between the BamHI and XhoI sites of the pMXs-IP vector.

### PCR Parameters

| | |
|---|---|
| Denature | 94° C. 2 min |
| Denature | 94° C. 15 sec* |
| Anneal | 56° C. 30 sec* |
| Extend | 68° C. 1 min* |
| Final extension | 68° C. 7 min |

*Repeat 22 cycles

The DNA Sequence of Mouse Mef2cM₃ (M₃ Domain is Underlined.)

(SEQ ID NO: 49)
atggggagaaaaaagattcagattacgaggataatggatgagcgtaac
agacaggtgacttttacgaagaggaaatttggattgatgaagaaggct
tatgagctgagcgtgctgtgcgactgtgagattgcactgatcatcttc
aacagcaccaacaagctgttccagtacgccagcactgacatggataag
gtgttgctcaagtacaccgagtacaacgagccgcacgagagccggaca
aactcagacattgtggagacattgagaaagaagggcctcaatggctgt
gacagcccagatcccgatgcagacgattcagtaggtcacagccctgag
tctgaggacaagtacaggaaaattaacgaagatattgatctaatgatc
agcaggcaaagattgtgtgctgttccacctcccagctttgagatgcca
gttaccatcccagtgtccagccataacagtttggtgtacagcaatcct
gtcagcacactgggaaaccccaatcttctgccactggcccacccgtct
ctgcagaggaatagtatgtctcctggtgtaacacatagacctccaagt
gcaggtaacacaggcggtctgatgggcggagatctgacatccggtgca
ggcaccagcgcagggaatggatacggcaacccccggaactcaccaggc
ctgctggtctcacctggtaacctgaacaagaatatacaagccaaatct
cctccccctatgaatctaggaatgaataatcgtaagccagatctccgc
gttcttatcccacctggcagcaagaacacgatgccatcagtgaatcaa
aggataaataactcccagtcggctcagtcattggctaccccggtggtt
tccgtagcaactcctactttaccaggacaaggaatgggaggatatcca
tcagccatttcaacaacatatggtactgagtactctctgagtagcgca
gatctgtcatctctgtctggcttcaacactgccagtgcgctccacctc
ggctctgtaactggctggcagcagcagcacctacataacatgccgcca
tctgccctcagtcagttgggagaccgtaccaccaccccttcgagatac
ccacaacacaccacgcgccacgaggcggggaggtctcctgttgacagc
ttgagcagctgtagcagttcctacgatgggagcgaccgagaggatcac
cggaacgaattccactcccccattggactcaccagaccttcgccggac
gaaagggaaagtccttcagtcaagcgcatgcgactctctgaaggatgg
gcaacaatggagcttctatcgccgccactccgggacatagacttgaca
ggccccgacggctctctctgctcctttgagacagcagacgacttctat
gatgatccgtgtttcgactcaccagacctgcgcttttttgaggacctg
gacccgcgcctggtgcacgtgggagccctcctgaaaccggaggagcac
tga

4) Human Mef2cM₃

The M₃ domain of the human MyoD cDNA was fused to the carboxy terminus of the full-length mouse Mef2c cDNA using PCR.

PCR for Human Mef2cM₃

The cDNA encoding the M₃ domain of human MyoD (amino acids 1-62) was amplified with the primer pair, hMEF2CM₃F1 (CTGAAGGATGGGCAACAATGGAGCTACTGTCGCC; SEQ ID NO: 13) and hMEF2CM₃R2 (CGACTCGAGGAATTCTCAGTGCTCTTCGGGTTTCAG; SEQ ID NO: 14). Full length MEF2C cDNA was amplified with the primer pair, Mef2cF3 (GAGGATCCGCCATGGGGAGAAAAAAG; SEQ ID NO: 11) and hMEF2CM₃R1 (TGGCGACAGTAGCTCCATTGTTGCCCATCCTTCAG; SEQ ID NO: 15). These PCR products were used as templates for the next PCR with the primer pair, Mef2cF3 and hMEF2CM₃R2. Mef2cM₃ was directly subcloned between the BamHI and XhoI sites of the pMXs-IP vector.

### PCR Parameters

| | |
|---|---|
| Denature | 94° C. 2 min |
| Denature | 94° C. 15 sec* |
| Anneal | 56° C. 30 sec* |
| Extend | 68° C. 1 min* |
| Final extension | 68° C. 7 min |

*Repeat 22 cycles

### The DNA Sequence of Human MEF2CM₃ (M₃ Domain is Underlined.)

(SEQ ID NO: 50)
atggggagaaaaaagattcagattacgaggattatggatgaacgtaacag
acaggtgacatttacaaagaggaaatttgggttgatgaagaaggcttatg
agctgagcgtgctgtgtgactgtgagattgcgctgatcatcttcaacagc
accaacaagctgttccagtatgccagcaccgacatggacaaagtgcttct
caagtacacggagtacaacgagccgcatgagagccggacaaactcagaca
tcgtggagacgttgagaaagaagggccttaatggctgtgacagcccagac
cccgatgcggacgattccgtaggtcacagccctgagtctgaggacaagta
caggaaaattaacgaagatattgatctaatgatcagcaggcaaagattgt
gtgctgttccacctcccaacttcgagatgccagtctccatcccagtgtcc
agccacaacagtttggtgtacagcaaccctgtcagctcactgggaaaccc
caacctattgccactggctcacccttctctgcagaggaatagtatgtctc
ctggtgtaacacatcgacctccaagtgcaggtaacacaggtggtctgatg
ggtggagacctcacgtctggtgcaggcaccagtgcagggaacgggtatgg
caatccccgaaactcaccaggtctgctggtctcacctggtaacttgaaca
agaatatgcaagcaaaatctcctcccccaatgaatttaggaatgaataac
cgtaaaccagatctccgagttcttattccaccaggcagcaagaatacgat
gccatcagtgtctgaggatgtcgacctgcttttgaatcaaaggataaata
actcccagtcggctcagtcattggctaccccagtggtttccgtagcaact
cctactttaccaggacaaggaatgggaggatatccatcagccatttcaac
aacatatggtaccgagtactctctgagtagtgcagacctgtcatctctgt
ctgggtttaacaccgccagcgctcttcaccttggttcagtaactggctgg
caacagcaacacctacataacatgccaccatctgccctcagtcagttggg
agcttgcactagcactcatttatctcagagttcaaatctctccctgcctt
ctactcaaagcctcaacatcaagtcagaacctgtttctcctcctagagac
cgtaccaccaccccttcgagatacccacaacacacgcgccacgaggcggg
gagatctcctgttgacagcttgagcagctgtagcagttcgtacgacggga
gcgaccgagaggatcaccggaacgaattccactcccccattggactcacc
agaccttcgccggacgaaagggaaagtccctcagtcaagcgcatgcgact
ttctgaaggatgggcaacaatggagctactgtcgccaccgctccgcgacg
tagacctgacggcccccgacggctctctctgctcctttgccacaacggac
gacttctatgacgacccgtgtttcgactccccggacctgcgcttcttcga
agacctggacccgcgcctgatgcacgtgggcgcgctcctgaaacccgaag
agcactga

5) Mouse Mef2cVP16

The cDNA encoding a part of the transactivation domain of VP16 cDNA was fused to the carboxy terminus of the full-length mouse Mef2c cDNA using PCR.

PCR for Mouse Mef2cVP16

The VP16 domain (amino acids 446-490) was prepared with PCR using the primer pair VP16F6 (CGAGGATCCGAATTCCTCGAGATGTTGGGGGACGGGGATTC; SEQ ID NO: 16) and VP16R6 (CGAGCGGCCGCTCACCCACCGTACTCGTCAATTC; SEQ ID NO: 17) and inserted into the BamHI and the NotI sites of the pMXs-IP vector to create the pMXs-IP VP16 vector. Mef2c was then PCR amplified with the primer pair Mef2cF3 (GAGGATCCGCCATGGGGAGAAAAAAG; SEQ ID NO: 11) and Mef2cR3 (CGACTCGAGTGTTGCCCATCCTTCAG; SEQ ID NO: 18), and inserted into the BamHI and XhoI site of pMXs-IP VP16 vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

The DNA Sequence of Mouse Mef2cVP16 (VP16 Domain is Underlined.)

(SEQ ID NO: 51)
atggggagaaaaaagattcagattacgaggataatggatgagcgtaacag
acaggtgacttttacgaagaggaaatttggattgatgaagaaggcttatg
agctgagcgtgctgtgcgactgtgagattgcactgatcatcttcaacagc
accaacaagctgttccagtacgccagcactgacatggataaggtgttgct
caagtacaccgagtacaacgagccgcacgagagccggacaaactcagaca
ttgtggagacattgagaaagaagggcctcaatggctgtgacagcccagat
cccgatgcagacgattcagtaggtcacagccctgagtctgaggacaagta
caggaaaattaacgaagatattgatctaatgatcagcaggcaaagattgt
gtgctgttccacctcccagctttgagatgccagttaccatcccagtgtcc
agccataacagtttggtgtacagcaatcctgtcagcacactgggaaaccc
caatcttctgccactggcccacccgtctctgcagaggaatagtatgtctc
ctggtgtaacacatagacctccaagtgcaggtaacacaggcggtctgatg
ggcggagatctgacatccggtgcaggcaccagcgcagggaatggatacgg
caacccccggaactcaccaggcctgctggtctcacctggtaacctgaaca
agaatatacaagccaaatctcctccccctatgaatctaggaatgaataat
cgtaagccagatctccgcgttcttatcccacctggcagcaagaacacgat
gccatcagtgaatcaaaggataaataactcccagtcggctcagtcattgg
ctaccccggtggtttccgtagcaactcctactttaccaggacaaggaatg
ggaggatatccatcagccatttcaacaacatatggtactgagtactctct
gagtagcgcagatctgtcatctctgtctggcttcaacactgccagtgcgc
tccacctcggctctgtaactggctggcagcagcagcacctacataacatg
ccgccatctgccctcagtcagttgggagaccgtaccaccaccccttcgag
atacccacaacacaccacgcgccacgaggcggggaggtctcctgttgaca
gcttgagcagctgtagcagttcctacgatgggagcgaccgagaggatcac
cggaacgaattccactcccccattggactcaccagaccttcgccggacga
aagggaaagtccttcagtcaagcgcatgcgactctctgaaggatgggcaa
cactcgagatgttgggggacggggattccccgggtccgggatttaccccc
cacgactccgccccctacggcgctctggatatggccgacttcgagtttga
gcagatgtttaccgatgcccttggaattgacgagtacggtgggtga

6) Human Mef2cVP16

The cDNA encoding a part of the transactivation domain of VP16 was fused to the carboxy terminus of the full-length mouse Mef2c cDNA using PCR.

PCR for Humans Mef2cVP16

The VP16 domain (amino acids 446-490) was prepared with PCR using the primer pair VP16F6 (CGAGGATCCGAATTCCTCGAGATGTTGGGGGACGGGGATTC; SEQ ID NO: 16) and VP16R6 (CGAGCGGCCGCTCACCCACCGTACTCGTCAATTC; SEQ ID NO: 17) and inserted into the BamHI and the NotI sites of the pMXs-IP vector to create the pMXs-IP VP16 vector. Mef2c was then PCR amplified with the primer pair Mef2cF3 (GAGGATCCGCCATGGGGAGAAAAAAG; SEQ ID NO: 11) and Mef2cR3 (CGACTCGAGTGTTGCCCATCCTTCAG; SEQ ID NO: 18), and inserted into the BamHI and XhoI site of pMXs-IP VP16 vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Human MEF2CVP16_(VP16 Domain is Underlined.)

(SEQ ID NO: 52)
atggggagaaaaaagattcagattacgaggattatggatgaacgtaac
agacaggtgacatttacaaagaggaaatttgggttgatgaagaaggct
tatgagctgagcgtgctgtgtgactgtgagattgcgctgatcatcttc
aacagcaccaacaagctgttccagtatgccagcaccgacatggacaaa
gtgcttctcaagtacacggagtacaacgagccgcatgagagccggaca
aactcagacatcgtggagacgttgagaaagaagggccttaatggctgt
gacagcccagaccccgatgcggacgattccgtaggtcacagccctgag
tctgaggacaagtacaggaaaattaacgaagatattgatctaatgatc
agcaggcaaagattgtgtgctgttccacctcccaacttcgagatgcca
gtctccatcccagtgtccagccacaacagtttggtgtacagcaaccct
gtcagctcactgggaaaccccaacctattgccactggctcacccttct
ctgcagaggaatagtatgtctcctggtgtaacacatcgacctccaagt
gcaggtaacacaggtggtctgatgggtggagacctcacgtctggtgca
ggcaccagtgcagggaacgggtatggcaatccccgaaactcaccaggt
ctgctggtctcacctggtaacttgaacaagaatatgcaagcaaaatct
cctcccccaatgaatttaggaatgaataaccgtaaaccagatctccga
gttcttattccaccaggcagcaagaatacgatgccatcagtgtctgag
gatgtcgacctgcttttgaatcaaaggataaataactcccagtcggct
cagtcattggctaccccagtggtttccgtagcaactcctactttacca
ggacaaggaatgggaggatatccatcagccatttcaacaacatatggt
accgagtactctctgagtagtgcagacctgtcatctctgtctgggttt
aacaccgccagcgctcttcaccttggttcagtaactggctggcaacag
caacacctacataacatgccaccatctgccctcagtcagttgggagct
tgcactagcactcatttatctcagagttcaaatctctccctgccttct
actcaaagcctcaacatcaagtcagaacctgtttctcctcctagagac
cgtaccaccaccccttcgagatacccacaacacacgcgccacgaggcg
gggagatctcctgttgacagcttgagcagctgtagcagttcgtacgac
gggagcgaccgagaggatcaccggaacgaattccactcccccattgga
ctcaccagaccttcgccggacgaaagggaaagtccctcagtcaagcgc
atgcgactttctgaaggatgggcaacactcgagatgttgggggacggg
gattccccgggtccgggatttaccccccacgactccgccccctacggc
gctctggatatggccgacttcgagtttgagcagatgtttaccgatgcc
cttggaattgacgagtacggtgggtga

### 7) Mouse M₃Gata4

The M₃ domain of the mouse MyoD cDNA was fused to the amino terminus of the full-length mouse Gata4 cDNA using PCR.

### PCR for Mouse M₃Gata4

The cDNA encoding the M₃ domain of mouse MyoD (amino acids 1-62) was amplified with the primer pair, MyoDOct4F4 (GAGAATTCGCCATGGAGCTTCTATCGCCGCCAC; SEQ ID NO: 19) and M₃Gata4R1 (GGCCAGGCTTTGGTACATGTGCTCCTCCGGTTTCAG; SEQ ID NO: 20). Full length Gata4 cDNA was amplified with the primer pair, M₃Gata4F1 (CTGAAACCGGAGGAGCACATGTACCAAAGCCTGGCC; SEQ ID NO: 21) and Gata4R1 (CGGAATTCTCTTACGCGGTGATTATGTC; SEQ ID NO: 22). These PCR products were used as templates for the next PCR with the primer pair, MyoDOct4F4 and Gata4R1. M₃Gata4 was directly subcloned into the EcoRI site of the pMXs-IP vector.

### PCR Parameters

| | | | |
|---|---|---|---|
| Denature | 94° C. | 2 | min |
| Denature | 94° C. | 15 | sec* |
| Anneal | 55° C. | 30 | sec* |
| Extend | 68° C. | 1 | min* |
| Final extension | 68° C. | 7 | min |

*Repeat 22 cycles

### The DNA Sequence of Mouse M₃Gata4 (M₃ Domain is Underlined.)

(SEQ ID NO: 53)
atggagcttctatcgccgccactccgggacatagacttgacaggcccc
gacggctctctctgctcctttgagacagcagacgacttctatgatgat
ccgtgtttcgactcaccagacctgcgcttttttgaggacctggacccg
cgcctggtgcacgtgggagccctcctgaaaccggaggagcacatgtac
caaagcctggccatggccgccaaccacggccccccgcccggcgcctac
gaagcaggtggccctggcgccttcatgcacagcgcgggcgccgcgtcc
tcgcccgtctacgtgcccactccgcgggtgccgtcctctgtgctgggc
ctgtcctacctgcagggcggtggcagtgccgctgcagctggaaccacc
tcgggtggcagctccggggccggcccgtcgggtgcagggcctgggacc
cagcagggtagccctggctggagccaagctggagccgagggagccgcc
tacaccccgccgcccgtgtccccgcgcttctctttcccggggactact
gggtccctggcggccgctgccgccgctgccgcagcccgggaagctgca
gcctacggcagtggcggcggggcggcgggcgctggtctggctggccga
gagcagtacgggcgtccgggcttcgccggctcctactccagcccctac
ccagcctacatggccgacgtgggagcatcctgggccgcagccgctgcc
gcctctgccggccccttcgacagcccagtcctgcacagcctgcctgga
cgggccaaccctggaagacaccccaatctcgatatgtttgatgacttc
tcagaaggcagagagtgtgtcaattgtggggccatgtccaccccactc
tggaggcgagatgggacgggacactacctgtgcaatgcctgtggcctc
tatcacaagatgaacggcatcaaccggcccctcattaagcctcagcgc
cgcctgtccgcttcccgccgggtaggcctctcctgtgccaactgccag
actaccaccaccacgctgtggcgtcgtaatgccgagggtgagcctgta
tgtaatgcctgcggcctctacatgaagctccatggggttcccaggcct
cttgcaatgcggaaggaggggattcaaaccagaaaacggaagcccaag
aacctgaataaatctaagacgccagcaggtcctgctggtgagaccctc
cctccctccagtggtgcctccagcggtaactccagcaatgccactagc
agcagcagcagcagtgaagagatgcgccccatcaagacagagcccggg
ctgtcatctcactatgggcacagcagctccatgtcccagacattcagt
actgtgtccggccacgggccctccatccatccagtgctgtctgctctg
aagctgtccccacaaggctatgcatctcctgtcactcagacatcgcag
gccagctccaagcaggactcttggaacagcctggtcctggctgacagt
catggggacataatcaccgcgtaa

### 8) Mouse Gata4M₃

The M₃ domain of the mouse MyoD cDNA was fused to the carboxy terminus of the full-length mouse Gata4 cDNA using PCR.

### PCR for Mouse Gata4M₃

The cDNA encoding the M₃ domain of mouse MyoD (amino acids 1-62) domain was prepared with PCR using the primer pair mM₃F1 (CGAGGATCCGAATTCCTCGAGATGGAGCTTCTATCGCCGCCAC; SEQ ID NO: 23) and mM₃R1 (CGAGCGGCCGCTCAGTGCTCCTCCGGTTTCAG; SEQ ID NO: 24) and inserted into the BamHI and the NotI sites of the pMXs-IP vector to create the pMXs-IP mM₃ vector. Gata4 was then PCR amplified with the primer pair Gata4F2 (CGAGGATCCGCCATGTACCAAAGCCTGGCC; SEQ ID NO: 25) and Gata4R2 (CGACTCGAGCGCGGTGATTATGTC; SEQ ID NO: 26), and inserted into the BamHI and XhoI site of pMXs-IP mM₃ vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Gata4M₃ (M₃ Domain is Underlined.)

(SEQ ID NO: 54)
atgtaccaaagcctggccatggccgccaaccacggccccccgcccggc
gcctacgaagcaggtggccctggcgccttcatgcacagcgcgggcgcc
gcgtcctcgcccgtctacgtgcccactccgcgggtgccgtcctctgtg
ctgggcctgtcctacctgcagggcggtggcagtgccgctgcagctgga
accacctcgggtggcagctccggggccggcccgtcgggtgcagggcct
gggacccagcagggtagccctggctggagccaagctggagccgaggga
gccgcctacaccccgccgcccgtgtccccgcgcttctctttcccgggg
actactgggtccctggcggccgctgccgccgctgccgcagcccgggaa
gctgcagcctacggcagtggcggcggggcggcgggcgctggtctggct
ggccgagagcagtacgggcgtccgggcttcgccggctcctactccagc
ccctacccagcctacatggccgacgtgggagcatcctgggccgcagcc
gctgccgcctctgccggccccttcgacagcccagtcctgcacagcctg
cctggacgggccaaccctggaagacaccccaatctcgatatgtttgat
gacttctcagaaggcagagagtgtgtcaattgtggggccatgtccacc
ccactctggaggcgagatgggacgggacactacctgtgcaatgcctgt
ggcctctatcacaagatgaacggcatcaaccggcccctcattaagcct
cagcgccgcctgtccgcttcccgccgggtaggcctctcctgtgccaac
tgccagactaccaccaccacgctgtggcgtcgtaatgccgagggtgag
cctgtatgtaatgcctgcggcctctacatgaagctccatggggttccc
aggcctcttgcaatgcggaaggaggggattcaaaccagaaaacggaag
cccaagaacctgaataaatctaagacgccagcaggtcctgctggtgag
accctccctccctccagtggtgcctccagcggtaactccagcaatgcc
actagcagcagcagcagcagtgaagagatgcgccccatcaagacagag
cccgggctgtcatctcactatgggcacagcagctccatgtcccagaca
ttcagtactgtgtccggccacgggccctccatccatccagtgctgtct
gctctgaagctgtccccacaaggctatgcatctcctgtcactcagaca
tcgcaggccagctccaagcaggactcttggaacagcctggtcctggct
gacagtcatggggacataatcaccgcgctcgagatggagcttctatcg
ccgccactccgggacatagacttgacaggccccgacggctctctctgc
tcctttgagacagcagacgacttctatgatgatccgtgtttcgactca
ccagacctgcgcttttttgaggacctggacccgcgcctggtgcacgtg
ggagccctcctgaaaccggaggagcactga

### 9) Mouse Gata4VP16

The cDNA encoding a part of the transactivation domain of VP16 cDNA was fused to the carboxy terminus of the full-length mouse Gata4 cDNA using PCR.

### PCR for Mouse Gata4VP16

The VP16 domain (amino acids 446-490) was prepared with PCR using the primer pair VP16F6 (CGAGGATCCGAATTCCTCGAGATGTTGGGGGACGGGGATTC; SEQ ID NO: 16) and VP16R6 (CGAGCGGCCGCTCACCCACCGTACTCGTCAATTC; SEQ ID NO: 17) and inserted into the BamHI and the NotI sites of the pMXs-IP vector to create the pMXs-IP VP16 vector.

Gata4 was then PCR amplified with the primer pair Gata4F2 (CGAGGATCCGCCATGTACCAAAGCCTGGCC; SEQ ID NO: 25) and Gata4R2 (CGACTCGAGCGCGGTGATTATGTC; SEQ ID NO: 26), and inserted into the BamHI and XhoI site of pMXs-IP VP16 vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Gata4VP16 (VP16 Domain is Underlined.)

(SEQ ID NO: 55)
atgtaccaaagcctggccatggccgccaaccacggccccccgcccggc
gcctacgaagcaggtggccctggcgccttcatgcacagcgcgggcgcc
gcgtcctcgcccgtctacgtgcccactccgcgggtgccgtcctctgtg
ctgggcctgtcctacctgcagggcggtggcagtgccgctgcagctgga
accacctcgggtggcagctccggggccggcccgtcgggtgcagggcct
gggacccagcagggtagccctggctggagccaagctggagccgaggga
gccgcctacaccccgccgcccgtgtccccgcgcttctctttcccgggg
actactgggtccctggcggccgctgccgccgctgccgcagcccgggaa
gctgcagcctacggcagtggcggcggggcggcgggcgctggtctggct
ggccgagagcagtacgggcgtccgggcttcgccggctcctactccagc
ccctacccagcctacatggccgacgtgggagcatcctgggccgcagcc
gctgccgcctctgccggccccttcgacagcccagtcctgcacagcctg
cctggacgggccaaccctggaagacaccccaatctcgatatgtttgat
gacttctcagaaggcagagagtgtgtcaattgtggggccatgtccacc
ccactctggaggcgagatgggacgggacactacctgtgcaatgcctgt
ggcctctatcacaagatgaacggcatcaaccggcccctcattaagcct
cagcgccgcctgtccgcttcccgccgggtaggcctctcctgtgccaac
tgccagactaccaccaccacgctgtggcgtcgtaatgccgagggtgag
cctgtatgtaatgcctgcggcctctacatgaagctccatggggttccc
aggcctcttgcaatgcggaaggaggggattcaaaccagaaaacggaag
cccaagaacctgaataaatctaagacgccagcaggtcctgctggtgag
accctccctccctccagtggtgcctccagcggtaactccagcaatgcc
actagcagcagcagcagcagtgaagagatgcgccccatcaagacagag
cccgggctgtcatctcactatgggcacagcagctccatgtcccagaca
ttcagtactgtgtccggccacgggccctccatccatccagtgctgtct
gctctgaagctgtccccacaaggctatgcatctcctgtcactcagaca
tcgcaggccagctccaagcaggactcttggaacagcctggtcctggct
gacagtcatggggacataatcaccgcgctcgagatgttgggggacggg
gattccccgggtccgggatttaccccccacgactccgccccctacggc
gctctggatatggccgacttcgagtttgagcagatgtttaccgatgcc
cttggaattgacgagtacggtgggtga

### 10) Mouse M₃Hand2

The M₃ domain of the human MyoD cDNA was fused to the amino terminus of the full-length mouse Hand2 cDNA using PCR.

### PCR for Mouse M₃Hand2

The cDNA encoding the M₃ domain of mouse MyoD (amino acids 1-62) was amplified with the primer pair, M₃F1 (CGAGGATCCGCCATGGAGCTTCTATCGCCGCCAC; SEQ ID NO: 27) and M₃R1 (CGACTCGAGGAATTCGTGCTCCTCCGGTTTCAG; SEQ ID NO: 28) by PCR and inserted between the BamHI and XhoI sites of the pMXs-IP vector to create the pMXsM₃-IP vector. Then the full-length mouse Hand2 cDNA was amplified with the primer pair, Hand2F1 (CGAGAATTCATGAGTCTGGTGGGGGGCTTTC; SEQ ID NO: 29) and Hand2R1 (GCGCTCGAGCTACTGCTTGAGCTCCAGGGC; SEQ ID NO: 30) by PCR and inserted between the EcoRI and XhoI sites of the pMXsM₃-IP vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse M₃Hand2 (M₃ Domain is Underlined.)

(SEQ ID NO: 56)
atggagcttctatcgccgccactccgggacatagacttgacaggcccc
gacggctctctctgctcctttgagacagcagacgacttctatgatgat
ccgtgtttcgactcaccagacctgcgcttttttgaggacctggacccg
cgcctggtgcacgtgggagccctcctgaaaccggaggagcacgaattc
atgagtctggtggggggctttccccaccaccccgtggtgcaccatgag
ggctacccattcgccgcagccgccgccgccgctgccgccgccgccgcc
agccgctgcagccacgaggagaacccctacttccacggctggcttatt
ggccacccggagatgtcgccccccgactacagtatggccctgtcctac
agccccgagtacgccagcggtgccgcgggcctggaccactcccattat
gggggagtgccgcccggcgccgggcctcccggcctgggggggccgcgc
ccggtgaagcgccggggcaccgccaaccgcaaggagcggcgcaggact
cagagcatcaacagcgccttcgccgagctgcgcgagtgcatccccaac
gtgcccgccgacaccaaactctccaagatcaagacactgcgcctggcc
accagctacatcgcctacctcatggatctgctggccaaggacgaccag
aacggagaggcggaggccttcaaggcggagatcaagaagaccgacgtg
aaagaggagaagaggaagaaagagctgaatgagatcttgaaaagcaca
gtgagcagcaacgacaagaaaaccaaaggccggacaggctggccacag
cacgtctgggccctggagctcaagcagtga

### 11) Mouse Hand2M₃

The M₃ domain of the human MyoD cDNA was fused to the carboxy terminus of the full-length mouse Hand2 cDNA using PCR.

### PCR for Mouse M₃Hand2

The cDNA encoding the M₃ domain of mouse MyoD (amino acids 1-62) domain was prepared with PCR using the primer pair mM₃F1 (CGAGGATCCGAATTCCTCGAGATGGAGCTTCTATCGCCGCCAC; SEQ ID NO: 23) and mM₃R1 (CGAGCGGCCGCTCAGTGCTCCTCCGGTTTCAG; SEQ ID NO: 24) and inserted into the BamHI and the NotI sites of the pMXs-IP vector to create the pMXs-IP mM₃ vector. Hand2 was then PCR amplified with the primer pair mHand2F2 (CGAGGATCCGCCATGAGTCTGGTGGGGGGCTTTC; SEQ ID NO: 31) and mHand2R2 (CGACTCGAGCTGCTTGAGCTCCAGGGC; SEQ ID NO: 32), and inserted into the BamHI and XhoI site of pMXs-IP mM₃ vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Hand2M₃ (M₃ Domain is Underlined.)

(SEQ ID NO: 57)
Atgagtctggtggggggctttccccaccaccccgtggtgcaccatgag
ggctacccattcgccgcagccgccgccgccgctgccgccgccgccgcc
agccgctgcagccacgaggagaacccctacttccacggctggcttatt
ggccacccggagatgtcgccccccgactacagtatggccctgtcctac
agccccgagtacgccagcggtgccgcgggcctggaccactcccattat
gggggagtgccgcccggcgccgggcctcccggcctgggggggccgcgc
ccggtgaagcgccggggcaccgccaaccgcaaggagcggcgcaggact
cagagcatcaacagcgccttcgccgagctgcgcgagtgcatccccaac
gtgcccgccgacaccaaactctccaagatcaagacactgcgcctggcc
accagctacatcgcctacctcatggatctgctggccaaggacgaccag
aacggagaggcggaggccttcaaggcggagatcaagaagaccgacgtg
aaagaggagaagaggaagaaagagctgaatgagatcttgaaaagcaca
gtgagcagcaacgacaagaaaaccaaaggccggacaggctggccacag
cacgtctgggccctggagctcaagcagctcgagatggagcttctatcg
ccgccactccgggacatagacttgacaggccccgacggctctctctgc
tcctttgagacagcagacgacttctatgatgatccgtgtttcgactca
ccagacctgcgcttttttgaggacctggacccgcgcctggtgcacgtg
ggagccctcctgaaaccggaggagcactga

### 12) Mouse Hand2VP16

The cDNA encoding a part of the transactivation domain of VP16 cDNA was fused to the carboxy terminus of the full-length mouse Hand2 cDNA using PCR.

### PCR for Mouse Gata4VP16

The VP16 domain (amino acids 446-490) was prepared with PCR using the primer pair VP16F6 (CGAGGATCCGAATTCCTCGAGATGTTGGGGGACGGGGATTC; SEQ ID NO: 16) and VP16R6 (CGAGCGGCCGCTCACCCACCGTACTCGTCAATTC; SEQ ID NO: 17) and inserted into the BamHI and the NotI sites of the pMXs-IP vector to create the pMXs-IP VP16 vector.

Hand2 was then PCR amplified with the primer pair mHand2F2 (CGAGGATCCGCCATGAGTCTGGTGGGGGGCTTTC; SEQ ID NO: 31) and mHand2R2 (CGACTCGAGCTGCTTGAGCTCCAGGGC; SEQ ID NO: 32), and inserted into the BamHI and XhoI site of pMXs-IP VP16 vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Hand2VP16 (VP16 Domain is Underlined.)

(SEQ ID NO: 58)
Atgagtctggtggggggctttccccaccaccccgtggtgcaccatgag
ggctacccattcgccgcagccgccgccgccgctgccgccgccgccgcc
agccgctgcagccacgaggagaacccctacttccacggctggcttatt
ggccacccggagatgtcgccccccgactacagtatggccctgtcctac
agccccgagtacgccagcggtgccgcgggcctggaccactcccattat
gggggagtgccgcccggcgccgggcctcccggcctgggggggccgcgc
ccggtgaagcgccggggcaccgccaaccgcaaggagcggcgcaggact
cagagcatcaacagcgccttcgccgagctgcgcgagtgcatccccaac
gtgcccgccgacaccaaactctccaagatcaagacactgcgcctggcc
accagctacatcgcctacctcatggatctgctggccaaggacgaccag
aacggagaggcggaggccttcaaggcggagatcaagaagaccgacgtg
aaagaggagaagaggaagaaagagctgaatgagatcttgaaaagcaca
gtgagcagcaacgacaagaaaaccaaaggccggacaggctggccacag
cacgtctgggccctggagctcaagcagctcgagatgttgggggacggg
gattccccgggtccgggatttaccccccacgactccgccccctacggc
gctctggatatggccgacttcgagtttgagcagatgtttaccgatgcc
cttggaattgacgagtacggtgggtga

### 13) Mouse M₃Tbx5

The M₃ domain of the mouse MyoD cDNA was fused to the amino terminus of the full-length mouse Tbx5 cDNA using PCR.

### PCR for Mouse M₃Tbx5

The cDNA encoding the M₃ domain of mouse MyoD (amino acids 1-62) was amplified with the primer pair, MyoDOct4F4 (GAGAATTCGCCATGGAGCTTCTATCGCCGCCAC; SEQ ID NO: 19) and M₃Tbx5R1 (CTCATCTGTATCGGCCATGTGCTCCTCCGGTTTCAG; SEQ ID NO: 33). Full length Tbx5 cDNA was amplified with the primer pair, M₃Tbx5F1 (CTGAAACCGGAGGAGCACATGGCCGATACAGATGAG; SEQ ID NO: 34) and Tbx5R1 (CGGAATTCTCTTAGCTATTCTCACTCCAC; SEQ ID NO: 35). These PCR products were used as templates for the next PCR with the primer set, MyoDOct4F4 and Tbx5R1. M₃Tbx5 was directly subcloned into the EcoRI site of the pMXs-IP vector.

### PCR Parameters

| | | | |
|---|---|---|---|
| Denature | 94° C. | 2 | min |
| Denature | 94° C. | 15 | sec* |
| Anneal | 55° C. | 30 | sec* |
| Extend | 68° C. | 1 | min* |
| Final extension | 68° C. | 7 | min |

*Repeat 22 cycles

### The DNA Sequence of Mouse M₃Tbx5 (M₃ Domain is Underlined.)

(SEQ ID NO: 59)
atggagcttctatcgccgccactccgggacatagacttgacaggcccc
gacggctctctctgctcctttgagacagcagacgacttctatgatgat
ccgtgtttcgactcaccagacctgcgcttttttgaggacctggacccg
cgcctggtgcacgtgggagccctcctgaaaccggaggagcacatggcc
gatacagatgagggctttggcctggcgcgcacgcctctggagcctgat
tccaaagacaggtcttgcgattcgaaacctgagagtgctctgggggct
cccagcaagtctccatcatccccgcaggctgccttcacccagcagggc
atggaaggaatcaaggtgtttcttcatgaacgtgaactgtggctgaag
ttccacgaagtgggcacagagatgatcatcaccaaggcagggaggaga
atgtttcctagttacaaagtgaaggtgactggccttaatcccaaaacg
aagtatattcttctcatggatattgttcccgcagacgaccacagatat
aaatttgctgataacaaatggtccgtaactggcaaagcagagcctgcc
atgccggggcgcctttacgtgcacccggactccccagcaaccggagcc
cactggatgcgacaacttgtctccttccagaagctcaaactcaccaac
aaccacctggacccgtttggacacattatcctgaactccatgcacaaa
taccagccccgattacacatcgtgaaagcagacgaaaataatgggttc
ggttcaaagaacactgcgttttgcacccacgtcttcccggagacagct
tttatcgctgtgacttcgtaccagaatcacaagatcacacagctgaaa
attgagaacaaccccttcgccaaaggctttcggggcagtgatgacctg
gagttacacaggatgtctcggatgcaaagtaaagagtatcctgtggtt
cccaggagcacagtgaggcacaaagtcacctccaaccacagccccttc
agcagcgagacccgagctctctccacctcatccaatttagggtcccag
taccagtgtgagaatggtgtctctggcccctcccaggaccttctgccc
ccacctaacccatacccactggcccaggagcacagccaaatttaccac
tgtaccaagaggaaagatgaggaatgttccagcacggagcacccctat
aagaagccgtacatggagacatcccccagcgaggaagacaccttctat
cgctcgggctacccccagcagcagggcctgagtacctcttacaggaca
gagtcggcccagcggcaggcctgcatgtatgccagctccgctcccccc
agcgagcccgtgcctagcctggaggacatcagctgtaacacatggccc
agcatgccctcctatagcagctgtaccgtcaccaccgtgcagcccatg
gaccgtcttccctaccagcacttctccgctcatttcacctcggggccc
ctggtccctcggttggctggcatggccaaccatggttctccccagctc
ggcgaagggatgtttcagcaccagacctcagtggcccatcagcctgtg
gtcaggcagtgcgggcctcagactggccttcagtctccgggcggcctc
cagcccccagagtttctctacactcacggcgtgcccaggaccctgtcc
ccccatcagtatcactcggtacacggcgtcggcatggtgccagagtgg
agtgagaatagctaa

### 14) Mouse Tbx5M₃

The M₃ domain of the human MyoD cDNA was fused to the carboxy terminus of the full-length mouse Tbx5 cDNA using PCR.

### PCR for Mouse Tbx5M₃

The cDNA encoding the M₃ domain of mouse MyoD (amino acids 1-62) domain was prepared with PCR using the primer pair mM₃F1 (CGAGGATCCGAATTCCTCGAGATGGAGCTTCTATCGCCGCCAC; SEQ ID NO: 23) and mM₃R1 (CGAGCGGCCGCTCAGTGCTCCTCCGGTTTCAG; SEQ ID NO: 24) and inserted into the BamHI and the NotI sites of the pMXs-IP vector to create the pMXs-IP mM₃ vector. Tbx5 was then PCR amplified with the primer pair Tbx5F2 (CGAGGATCCGCCATGGCCGATACAGAG; SEQ ID NO: 36) and Tbx5R2 (CGACTCGAGGCTATTCTCACTCCAC; SEQ ID NO: 37), and inserted into the BamHI and XhoI site of pMXs-IP mM₃ vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Tbx5M₃ (M₃ Domain is Underlined.)

(SEQ ID NO: 60)
atggccgatacagatgagggctttggcctggcgcgcacgcctctggag
cctgattccaaagacaggtcttgcgattcgaaacctgagagtgctctg
ggggctcccagcaagtctccatcatccccgcaggctgccttcacccag
cagggcatggaaggaatcaaggtgtttcttcatgaacgtgaactgtgg
ctgaagttccacgaagtgggcacagagatgatcatcaccaaggcaggg
aggagaatgtttcctagttacaaagtgaaggtgactggccttaatccc
aaaacgaagtatattcttctcatggatattgttcccgcagacgaccac
agatataaatttgctgataacaaatggtccgtaactggcaaagcagag
cctgccatgccggggcgcctttacgtgcacccggactccccagcaacc
ggagcccactggatgcgacaacttgtctccttccagaagctcaaactc
accaacaaccacctggacccgtttggacacattatcctgaactccatg
cacaaataccagccccgattacacatcgtgaaagcagacgaaaataat
gggttcggttcaaagaacactgcgttttgcacccacgtcttcccggag
acagcttttatcgctgtgacttcgtaccagaatcacaagatcacacag
ctgaaaattgagaacaaccccttcgccaaaggctttcggggcagtgat
gacctggagttacacaggatgtctcggatgcaaagtaaagagtatcct
gtggttcccaggagcacagtgaggcacaaagtcacctccaaccacagc
cccttcagcagcgagacccgagctctctccacctcatccaatttaggg
tcccagtaccagtgtgagaatggtgtctctggcccctcccaggacctt
ctgcccccacctaacccatacccactggcccaggagcacagccaaatt
taccactgtaccaagaggaaagatgaggaatgttccagcacggagcac
ccctataagaagccgtacatggagacatcccccagcgaggaagacacc
ttctatcgctcgggctacccccagcagcagggcctgagtacctcttac
aggacagagtcggcccagcggcaggcctgcatgtatgccagctccgct
ccccccagcgagcccgtgcctagcctggaggacatcagctgtaacaca
tggcccagcatgccctcctatagcagctgtaccgtcaccaccgtgcag
cccatggaccgtcttccctaccagcacttctccgctcatttcacctcg
gggcccctggtccctcggttggctggcatggccaaccatggttctccc
cagctcggcgaagggatgtttcagcaccagacctcagtggcccatcag
cctgtggtcaggcagtgcgggcctcagactggccttcagtctccgggc
ggcctccagcccccagagtttctctacactcacggcgtgcccaggacc
ctgtccccccatcagtatcactcggtacacggcgtcggcatggtgcca
gagtggagtgagaatagcctcgagatggagcttctatcgccgccactc
cgggacatagacttgacaggccccgacggctctctctgctcctttgag
acagcagacgacttctatgatgatccgtgtttcgactcaccagacctg
cgcttttttgaggacctggacccgcgcctggtgcacgtgggagccctc
ctgaaaccggaggagcactga

### 15) Mouse Tbx5VP16

The cDNA encoding a part of the transactivation domain of VP16 cDNA was fused to the carboxy terminus of the full-length mouse Tbx5 cDNA using PCR.

### PCR for Mouse Tbx5VP16

The VP16 domain (amino acids 446-490) was prepared with PCR using the primer pair VP16F6 (CGAGGATCCGAATTCCTCGAGATGTTGGGGGACGGGGATTC; SEQ ID NO: 16) and VP16R6 (CGAGCGGCCGCTCACCCACCGTACTCGTCAATTC; SEQ ID NO: 17) and inserted into the BamHI and the NotI sites of the pMXs-IP vector to create the pMXs-IP VP16 vector.

Tbx5 was then PCR amplified with the primer pair Tbx5F2 (CGAGGATCCGCCATGGCCGATACAGAG; SEQ ID NO: 36) and Tbx5R2 (CGACTCGAGGCTATTCTCACTCCAC; SEQ ID NO: 37), and inserted into the BamHI and XhoI site of pMXs-IP VP16 vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse Tbx5VP16_(VP16 Domain is Underlined.)

(SEQ ID NO: 61)
atggccgatacagatgagggctttggcctggcgcgcacgcctctggag
cctgattccaaagacaggtcttgcgattcgaaacctgagagtgctctg
ggggctcccagcaagtctccatcatccccgcaggctgccttcacccag
cagggcatggaaggaatcaaggtgtttcttcatgaacgtgaactgtgg
ctgaagttccacgaagtgggcacagagatgatcatcaccaaggcaggg
aggagaatgtttcctagttacaaagtgaaggtgactggccttaatccc
aaaacgaagtatattcttctcatggatattgttcccgcagacgaccac
agatataaatttgctgataacaaatggtccgtaactggcaaagcagag
cctgccatgccggggcgcctttacgtgcacccggactccccagcaacc
ggagcccactggatgcgacaacttgtctccttccagaagctcaaactc
accaacaaccacctggacccgtttggacacattatcctgaactccatg
cacaaataccagccccgattacacatcgtgaaagcagacgaaaataat
gggttcggttcaaagaacactgcgttttgcacccacgtcttcccggag
acagcttttatcgctgtgacttcgtaccagaatcacaagatcacacag
ctgaaaattgagaacaaccccttcgccaaaggctttcggggcagtgat
gacctggagttacacaggatgtctcggatgcaaagtaaagagtatcct
gtggttcccaggagcacagtgaggcacaaagtcacctccaaccacagc
cccttcagcagcgagacccgagctctctccacctcatccaatttaggg
tcccagtaccagtgtgagaatggtgtctctggcccctcccaggacctt
ctgcccccacctaacccatacccactggcccaggagcacagccaaatt
taccactgtaccaagaggaaagatgaggaatgttccagcacggagcac
ccctataagaagccgtacatggagacatcccccagcgaggaagacacc
ttctatcgctcgggctacccccagcagcagggcctgagtacctcttac
aggacagagtcggcccagcggcaggcctgcatgtatgccagctccgct
ccccccagcgagcccgtgcctagcctggaggacatcagctgtaacaca
tggcccagcatgccctcctatagcagctgtaccgtcaccaccgtgcag
cccatggaccgtcttccctaccagcacttctccgctcatttcacctcg
gggcccctggtccctcggttggctggcatggccaaccatggttctccc
cagctcggcgaagggatgtttcagcaccagacctcagtggcccatcag
cctgtggtcaggcagtgcgggcctcagactggccttcagtctccgggc
ggcctccagcccccagagtttctctacactcacggcgtgcccaggacc
ctgtccccccatcagtatcactcggtacacggcgtcggcatggtgcca
gagtggagtgagaatagcctcgagatgttgggggacggggattccccg
ggtccgggatttaccccccacgactccgccccctacggcgctctggat
atggccgacttcgagtttgagcagatgtttaccgatgcccttggaatt
gacgagtacggtgggtga

### 16) Human M₃ETS2

The M₃ domain of the human MyoD cDNA was fused to the amino terminus of the full-length human ETS2 cDNA using PCR.

### PCR for Human M₃ETS2

The cDNA encoding the M₃ domain of human MyoD (amino acids 1-62) was amplified with the primer pair, hM₃F5 (CGAGGATCCATGGAGCTACTGTCGCCAC; SEQ ID NO:38) and hM₃R4 (CGACTCGAGGAATTCGTGCTCTTCGGGTTTCAG; SEQ ID NO:39) by PCR and subcloned between the BamHI and XhoI sites of the pMXs-IP vector to create the pMXshM₃-IP vector. Then, the full-length human ETS2 cDNA was amplified the primer pair, hETS2F1 (CGAGAATTCATGAATGATTTCGGAATCAAG; SEQ ID NO: 40) and hETS2R1 (CGACTCGAGTCAGTCCTCCGTGTCGGGC; SEQ ID NO: 41) by PCR and subcloned between the EcoRI and XhoI sites of the pMXshM₃-IP vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Human M₃ETS2 (M₃ Domain is Underlined.)

(SEQ ID NO: 62)
atggagctactgtcgccaccgctccgcgacgtagacctgacggccccc
gacggctctctctgctcctttgccacaacggacgacttctatgacgac
ccgtgtttcgactccccggacctgcgcttcttcgaagacctggacccg
cgcctgatgcacgtgggcgcgctcctgaaacccgaagagcacgaattc
atgaatgatttcggaatcaagaatatggaccaggtagcccctgtggct
aacagttacagagggacactcaagcgccagccagcctttgacaccttt
gatgggtccctgtttgctgtttttccttctctaaatgaagagcaaaca
ctgcaagaagtgccaacaggcttggattccatttctcatgactccgcc
aactgtgaattgcctttgttaaccccgtgcagcaaggctgtgatgagt
caagccttaaaagctaccttcagtggcttcaaaaaggaacagcggcgc
ctgggcattccaaagaacccctggctgtggagtgagcaacaggtatgc
cagtggcttctctgggccaccaatgagttcagtctggtgaacgtgaat
ctgcagaggttcggcatgaatggccagatgctgtgtaaccttggcaag
gaacgctttctggagctggcacctgactttgtgggtgacattctctgg
gaacatctggagcaaatgatcaaagaaaaccaagaaaagacagaagat
caatatgaagaaaattcacacctcacctccgttcctcattggattaac
agcaatacattaggttttggcacagagcaggcgccctatggaatgcag
acacagaattaccccaaaggcggcctcctggacagcatgtgtccggcc
tccacacccagcgtactcagctctgagcaggagtttcagatgttcccc
aagtctcggctcagctccgtcagcgtcacctactgctctgtcagtcag
gacttcccaggcagcaacttgaatttgctcaccaacaattctgggact
cccaaagaccacgactcccctgagaacggtgcggacagcttcgagagc
tcagactccctcctccagtcctggaacagccagtcgtccttgctggat
gtgcaacgggttccttccttcgagagcttcgaagatgactgcagccag
tctctctgcctcaataagccaaccatgtctttcaaggattacatccaa
gagaggagtgacccagtggagcaaggcaaaccagttatacctgcagct
gtgctggccggcttcacaggaagtggacctattcagctgtggcagttt
ctcctggagctgctatcagacaaatcctgccagtcattcatcagctgg
actggagacggatgggagtttaagctcgccgaccccgatgaggtggcc
cgccggtggggaaagaggaaaaataagcccaagatgaactacgagaag
ctgagccggggcttacgctactattacgacaagaacatcatccacaag
acgtcggggaagcgctacgtgtaccgcttcgtgtgcgacctccagaac
ttgctggggttcacgcccgaggaactgcacgccatcctgggcgtccag
cccgacacggaggactga

### 17) Human M₃MESP1

The M₃ domain of the human MyoD cDNA was fused to the amino terminus of the full-length human MESP1 cDNA using PCR.

### PCR for Human M₃MESP1 (M₃ Domain is Underlined.)

The cDNA encoding the M₃ domain of human MyoD (amino acids 1-62) was amplified with the primer pair, hM₃F5 (CGAGGATCCATGGAGCTACTGTCGCCAC; SEQ ID NO:38) and hM₃R4 (CGACTCGAGGAATTCGTGCTCTTCGGGTTTCAG; SEQ ID NO:39) by PCR and subcloned between the BamHI and XhoI sites of the pMXs-IP vector to create the pMXshM₃-IP vector. Then, the full-length human MESP1 cDNA was amplified with the primer pair, hMESP1F1 (CGAGAATTCATGGCCCAGCCCCTGTGCCCG; SEQ ID NO: 42) and hMESP1R1 (CGACTCGAGTCACTTGGGCTCCTCAGGC; SEQ ID NO: 43) by PCR and subcloned between the EcoRI and XhoI sites of the pMXshM₃-IP vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Human M₃MESP1 (M₃ Domain is Underlined.)

(SEQ ID NO: 63)
Atggagctactgtcgccaccgctccgcgacgtagacctgacggccccc
gacggctctctctgctcctttgccacaacggacgacttctatgacgac
ccgtgtttcgactccccggacctgcgcttcttcgaagacctggacccg
cgcctgatgcacgtgggcgcgctcctgaaacccgaagagcacgaattc
atggcccagcccctgtgcccgccgctctccgagtcctggatgctctct
gcggcctggggcccaactcggcggccgccgccctccgacaaggactgc
ggccgctccctcgtctcgtccccagactcatggggcagcaccccagcc
gacagccccgtggcgagccccgcgcggccaggcaccctccgggacccc
cgcgccccctccgtaggtaggcgcggcgcgcgcagcagccgcctgggc
agcgggcagaggcagagcgccagtgagcgggagaaactgcgcatgcgc
acgctggcccgcgccctgcacgagctgcgccgctttctaccgccgtcc
gtggcgcccgcgggccagagcctgaccaagatcgagacgctgcgcctg
gctatccgctatatcggccacctgtcggccgtgctaggcctcagcgag
gagagtctccagcgccggtgccggcagcgcggtgacgcggggtcccct
cggggctgcccgctgtgccccgacgactgccccgcgcagatgcagaca
cggacgcaggctgaggggcaggggcaggggcgcgggctgggcctggta
tccgccgtccgcgccggggcgtcctggggatccccgcctgcctgcccc
ggagcccgagctgcacccgagccgcgcgacccgcctgcgctgttcgcc
gaggcggcgtgccctgaagggcaggcgatggagccaagcccaccgtcc
ccgctccttccgggcgacgtgctggctctgttggagacctggatgccc
ctctcgcctctggagtggctgcctgaggagcccaagtga

### 18) Mouse VP16Mef2c

The cDNA encoding a part of the transactivation domain of VP16 was fused to the amino terminus of the mouse full-length Mef2c cDNA using PCR.

### PCR for VP16Mef2c

The cDNA encoding a part of the transactivation domain of VP16 (amino acids 446-490) was amplified with the primer pair, V16F5 (CGAGGATCCATGTTGGGGGACGGGGATTC; SEQ ID NO: 44) and V16Merf2cR1 (CTTTTTTCTCCCCATCCCACCGTACTCGTC; SEQ ID NO: 45). Full length Mef2c cDNA was amplified with the primer pair, VP16Mef2cF1 (GACGAGTACGGTGGGATGGGGAGAAAAAAG; SEQ ID NO: 46) and Mef2cR2 (CGCTCGAGTCTCATGTTGCCCATCCTTCAG; SEQ ID NO: 4). These PCR products were used as templates for the next PCR with the primer pair, V16F5 and Mef2cR2. VP16Mef2c was directly subcloned between the BamHI and XhoI sites of pMXs-IP vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Mouse VP16Mef2c (VP16 Domain is Underlined.)

(SEQ ID NO: 64)
atgttgggggacggggattccccgggtccgggatttaccccccacgac
tccgccccctacggcgctctggatatggccgacttcgagtttgagcag
atgtttaccgatgcccttggaattgacgagtacggtgggatggggaga
aaaaagattcagattacgaggataatggatgagcgtaacagacaggtg
acttttacgaagaggaaatttggattgatgaagaaggcttatgagctg
agcgtgctgtgcgactgtgagattgcactgatcatcttcaacagcacc
aacaagctgttccagtacgccagcactgacatggataaggtgttgctc
aagtacaccgagtacaacgagccgcacgagagccggacaaactcagac
attgtggagacattgagaaagaagggcctcaatggctgtgacagccca
gatcccgatgcagacgattcagtaggtcacagccctgagtctgaggac
aagtacaggaaaattaacgaagatattgatctaatgatcagcaggcaa
agattgtgtgctgttccacctcccagctttgagatgccagttaccatc
ccagtgtccagccataacagtttggtgtacagcaatcctgtcagcaca
ctgggaaaccccaatcttctgccactggcccacccgtctctgcagagg
aatagtatgtctcctggtgtaacacatagacctccaagtgcaggtaac
acaggcggtctgatgggcggagatctgacatccggtgcaggcaccagc
gcagggaatggatacggcaacccccggaactcaccaggcctgctggtc
tcacctggtaacctgaacaagaatatacaagccaaatctcctccccct
atgaatctaggaatgaataatcgtaagccagatctccgcgttcttatc
ccacctggcagcaagaacacgatgccatcagtgaatcaaaggataaat
aactcccagtcggctcagtcattggctaccccggtggtttccgtagca
actcctactttaccaggacaaggaatgggaggatatccatcagccatt
tcaacaacatatggtactgagtactctctgagtagcgcagatctgtca
tctctgtctggcttcaacactgccagtgcgctccacctcggctctgta
actggctggcagcagcagcacctacataacatgccgccatctgccctc
agtcagttgggagcttgcactagcactcatttatctcagagttcaaat
ctctccctgccttctactcaaagcctcagcatcaagtcagaacctgtt
tctcctcctagagaccgtaccaccaccccttcgagatacccacaacac
accacgcgccacgaggcggggaggtctcctgttgacagcttgagcagc
tgtagcagttcctacgatgggagcgaccgagaggatcaccggaacgaa
ttccactcccccattggactcaccagaccttcgccggacgaaagggaa
agtccttcagtcaagcgcatgcgactctctgaaggatgggcaacatga

### 19) Human VP16Mef2c

The cDNA encoding a part of the transactivation domain of VP16 was fused to the amino terminus of the human full-length Mef2c cDNA using PCR.

### PCR for VP16Mef2c

The cDNA encoding a part of the transactivation domain of VP16 (amino acids 446-490) was amplified with the primer pair, V16F5 (CGAGGATCCATGTTGGGGGACGGGGATTC; SEQ ID NO: 44) and V16Merf2cR1 (CTTTTTTCTCCCCATCCCACCGTACTCGTC; SEQ ID NO: 45). Full length Mef2c cDNA was amplified with the primer pair, VP16Mef2cF1 (GACGAGTACGGTGGGATGGGGAGAAAAAAG; SEQ ID NO: 46) and hMEF2CR1 (CGACTCGAGTTATGTTGCCCATCCTTC; SEQ ID NO: 8). These PCR products were used as templates for the next PCR with the primer pair, V16F5 and Mef2cR2. VP16Mef2c was directly subcloned between the BamHI and XhoI sites of pMXs-IP vector.

### PCR Parameters

| | | | |
|---|---|---|---|
| Denature | 94° C. | 2 | min |
| Denature | 94° C. | 15 | sec* |
| Anneal | 56° C. | 30 | sec* |
| Extend | 68° C. | 1 | min* |
| Final extension | 68° C. | 7 | min |

*Repeat 22 cycles

### The DNA Sequence of Human VP16Mef2c (VP16 Domain is Underlined.)

(SEQ ID NO: 65)
atgttgggggacggggattccccgggtccgggatttaccccccacgac
tccgccccctacggcgctctggatatggccgacttcgagtttgagcag
atgtttaccgatgcccttggaattgacgagtacggtgggatggggaga
aaaaagattcagattacgaggattatggatgaacgtaacagacaggtg
acatttacaaagaggaaatttgggttgatgaagaaggcttatgagctg
agcgtgctgtgtgactgtgagattgcgctgatcatcttcaacagcacc
aacaagctgttccagtatgccagcaccgacatggacaaagtgcttctc
aagtacacggagtacaacgagccgcatgagagccggacaaactcagac
atcgtggagacgttgagaaagaagggccttaatggctgtgacagccca
gaccccgatgcggacgattccgtaggtcacagccctgagtctgaggac
aagtacaggaaaattaacgaagatattgatctaatgatcagcaggcaa
agattgtgtgctgttccacctcccaacttcgagatgccagtctccatc
ccagtgtccagccacaacagtttggtgtacagcaaccctgtcagctca
ctgggaaaccccaacctattgccactggctcacccttctctgcagagg
aatagtatgtctcctggtgtaacacatcgacctccaagtgcaggtaac
acaggtggtctgatgggtggagacctcacgtctggtgcaggcaccagt
gcagggaacgggtatggcaatccccgaaactcaccaggtctgctggtc
tcacctggtaacttgaacaagaatatgcaagcaaaatctcctccccca
atgaatttaggaatgaataaccgtaaaccagatctccgagttcttatt
ccaccaggcagcaagaatacgatgccatcagtgtctgaggatgtcgac
ctgcttttgaatcaaaggataaataactcccagtcggctcagtcattg
gctaccccagtggtttccgtagcaactcctactttaccaggacaagga
atgggaggatatccatcagccatttcaacaacatatggtaccgagtac
tctctgagtagtgcagacctgtcatctctgtctgggtttaacaccgcc
agcgctcttcaccttggttcagtaactggctggcaacagcaacaccta
cataacatgccaccatctgccctcagtcagttgggagcttgcactagc
actcatttatctcagagttcaaatctctccctgccttctactcaaagc
ctcaacatcaagtcagaacctgtttctcctcctagagaccgtaccacc
accccttcgagatacccacaacacacgcgccacgaggcggggagatct
cctgttgacagcttgagcagctgtagcagttcgtacgacgggagcgac
cgagaggatcaccggaacgaattccactcccccattggactcaccaga
ccttcgccggacgaaagggaaagtccctcagtcaagcgcatgcgactt
tctgaaggatgggcaacataa

### BIBLIOGRAPHY

Ieda, M., et al. (2010). Cell 142, 375-386.
Song, K., et al. (2012). Nature 485, 599-604.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for preparing a cardiovascular cell comprising introducing a nucleic acid sequence which is a fusion of a heterologous transactivation domain and a transcription factor into a cell or tissue.

2. The method of claim 1, wherein the fusion is a DNA, RNA or protein fusion.

3. The method of claim 1, wherein the fusion is introduced to the cell in vivo or in vitro.

4. The method of claim 1, wherein the transcription factor is selected from Gata4, Mef2c, Tbx5, Hand2, Ets2, Mesp1, or Gata2.

5. The method of claim 1, wherein the transactivation domain is obtained from MyoD or VP16 or other transcription factor.

6. The method of claim 1, wherein the transactivation domain is obtained from MyoD.

7. The method of claim 5, wherein the transactivation domain of MyoD comprises an N-terminus region of MyoD.

8. The method of claim 1, wherein the transactivation domain comprises amino acids 1-62 of MyoD or is at least 80% identical thereto.

9. The method of claim 1, wherein the cell is mammalian.

10. The method of claim 9, wherein the mammalian cell is human.

11. The method of claim 1, wherein the non-cardiovascular cell is a somatic cell.

12. The method of claim 11, wherein the somatic cell is a fibroblast cell and/or a non-fibroblast cell of any tissue origin, including a keratinocyte, myocyte, satellite cell, tendon cell, chondrocyte, osteocyte, adipocyte, endothelial cell, angioblast, mesoangioblast, pericyte, mural cell, mesangial cell, juxtaglomerular cell, macula densa cell, stromal cell, epidermal cell, blood cell, bone marrow cell, germ cell, or any type of a stem cell.

13. A method comprising: (1) isolating and collecting a somatic cell from a subject; (2) inducing said somatic cell from the subject into a cardiovascular cell according to the method of claim 1 and (3) administering an effective amount of the cardiovascular cell to the subject in need thereof.

14. The method of claim 13, wherein the somatic cell is a fibroblast cell and/or a non-fibroblast cell of any tissue origin, including a keratinocyte, myocyte, satellite cell, tendon cell, chondrocyte, osteocyte, adipocyte, endothelial cell, angioblast, mesoangioblast, pericyte, mural cell, mesangial cell, juxtaglomerular cell, macula densa cell, stromal cell, epidermal cell, blood cell, bone marrow cell, germ cell, or any type of a stem cell.

15. The method of claim 1, wherein the cardiovascular cell are cardiomyocytes, endothelial cells and/or smooth muscle cells.
